(19) United States
(12) Patent Application Publication
(10) Pub. No.: US 2026/0253689 A1
(43) Pub. Date: Aug. 27, 2026

(54) SYSTEM

(71) Applicant: SoftBank Group Corp., Tokyo (JP)

(72) Inventor: Seiwa KOBAYASHI, Tokyo (JP)

(73) Assignee: SoftBank Group Corp., Tokyo (JP)

(21) Appl. No.: 19/534,967

(22) Filed: Feb. 10, 2026

(30) Foreign Application Priority Data
Feb. 21, 2025 (JP) 2025-027081

(51) Int. Cl. G16H 10/60; G06Q 10/10

(52) U.S. Cl. CPC G16H 10/60; G06Q 10/10

(57) ABSTRACT

The system according to the embodiment comprises a reception unit, a generation unit, a selection unit, and a feedback unit. The reception unit inputs a patient's symptoms or health information. The generation unit analyzes the information input by the reception unit and generates a treatment plan. The selection unit selects a clinic based on the treatment plan generated by the generation unit. The feedback unit provides feedback on the progress of treatment at the clinic selected by the selection unit.

### CROSS-REFERENCE TO RELATED APPLICATION

The present application claims priority to and incorporates by reference the entire contents of Japanese Patent Application No. 2025-027081 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, the process of providing an optimal treatment plan based on a patient's symptoms or health information and selecting an appropriate clinic is complex and has room for improvement.

### SUMMARY OF THE INVENTION

The system according to the embodiment comprises a reception unit, a generation unit, a selection unit, and a feedback unit. The reception unit inputs a patient's symptoms or health information. The generation unit analyzes the information input by the reception unit and generates a treatment plan. The selection unit selects a clinic based on the treatment plan generated by the generation unit. The feedback unit provides feedback on the progress of treatment at the clinic selected by the selection unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

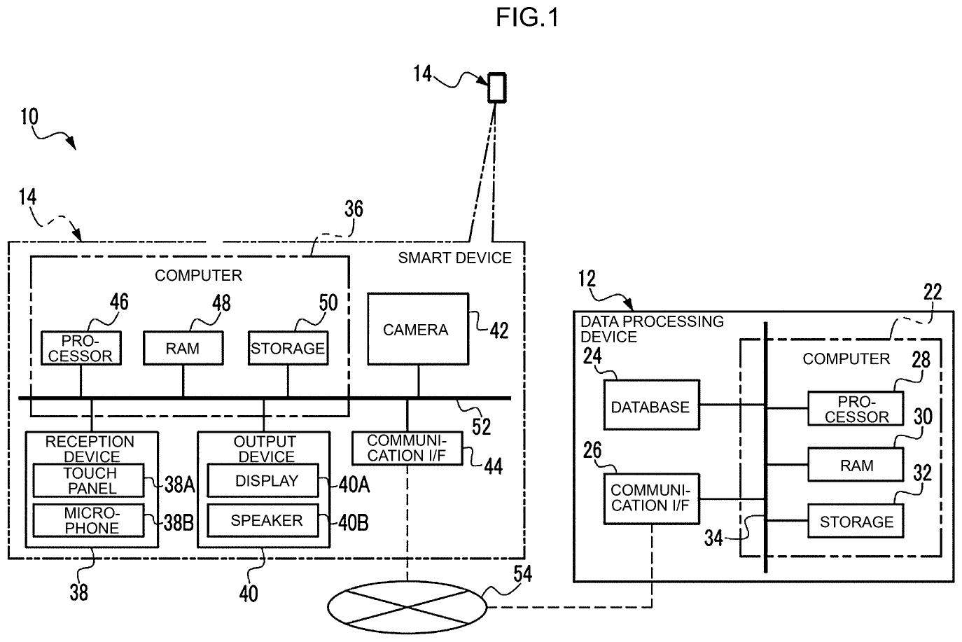

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

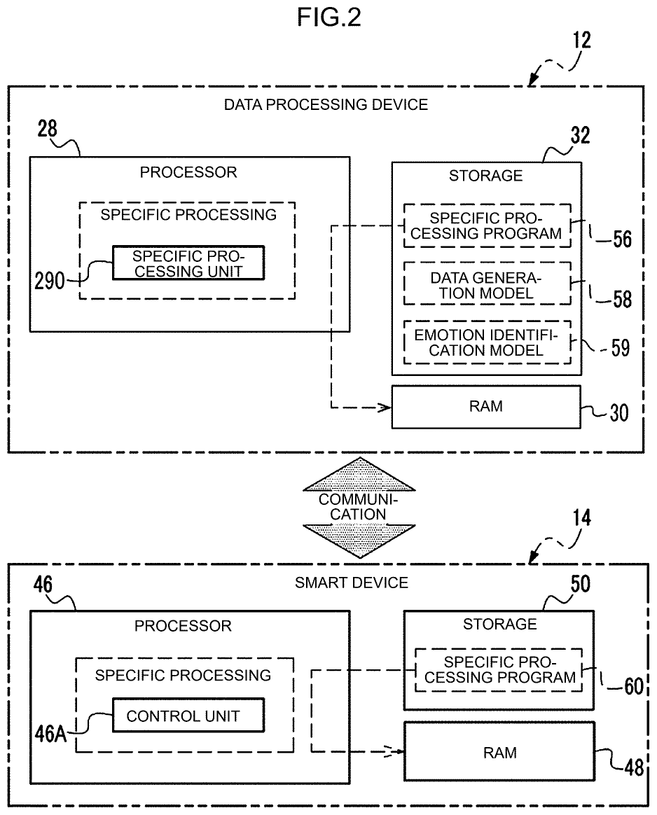

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The service for low back pain patients according to the embodiment of the present invention is a system that utilizes AI technology to provide individualized treatment plans based on the patient's symptoms and health information. This system enables patients to select the optimal clinic for themselves and receive a customized treatment plan. Furthermore, simple online access and feedback functions allow the progress of treatment to be shared in real time. For example, a patient inputs their symptoms and health information. At this time, the patient enters detailed information such as the degree of low back pain, onset timing, past treatment history, and lifestyle. For instance, information such as what movements worsen the low back pain, what treatments have been received, and what kind of exercise is performed daily is input. This information is input into the AI. Next, the AI analyzes the input information. Based on the patient's symptoms and health information, the AI generates an optimal treatment plan. For example, if the cause of the patient's low back pain is muscle tension, the AI proposes a treatment plan centered on stretching and massage. In addition, the AI considers the patient's lifestyle and past treatment history to generate a customized treatment plan. Based on the generated treatment plan, the patient can select the optimal clinic for themselves. For example, based on the treatment plan proposed by the AI, the patient can select the optimal clinic from nearby clinics. By referring to clinic ratings and reviews, more appropriate choices can be made. Furthermore, simple online access and feedback functions allow the progress of treatment to be shared in real time. For example, after receiving treatment, the patient can provide feedback on the effect and impressions online. This enables real-time confirmation of treatment effects and modification of the treatment plan as needed. Through this mechanism, low back pain patients can receive the best treatment suited to themselves. Since the patient receives the optimal treatment based on the individualized treatment plan generated by the AI, improvement of symptoms can be expected. In addition, the online feedback function allows the progress of treatment to be shared in real time, maximizing the effectiveness of treatment. For example, by providing feedback after receiving treatment, the AI can modify the treatment plan and provide more effective treatment. As a result, the service for low back pain patients can provide individualized treatment plans based on the patient's symptoms and health information, select the optimal clinic, and provide feedback on the progress of treatment. Specifically, the system is composed of multiple modules such as a reception unit, a generation unit, a selection unit, and a feedback unit. The system preprocesses the symptoms and health information input by the patient at the reception unit (e.g., entering the degree of low back pain as a numerical value on a scale of 0 to 10, entering the onset timing in date format, entering past treatment history as text or selectable options, entering lifestyle as category selection or free description) as multidimensional vectors (e.g., numerical vectors, categorical encoding, text embedding vectors, etc.) and inputs them to the generation unit. The generation unit uses AI models such as multilayer perceptrons, Transformer-based large language models, or convolutional neural networks for symptom classification to generate candidate treatment plans from the input vectors. Examples of AI model inputs include (1) low back pain score=7, onset date=2024-05-01, past treatment history=“acupuncture, chiropractic”, lifestyle=“mainly desk work”; and (2) low back pain score=3, onset date=2023-12-15, past treatment history=“exercise therapy”, lifestyle=“sports enthusiast”. The AI model outputs structured data for the treatment plan (e.g., treatment content labels, recommended frequency, recommended duration, recommended clinic type, estimated effect score, etc.), such as “stretching: 3 times a week, massage: twice a month, recommended clinic type: chiropractic clinic, estimated effect: high” or “physical therapy: once a week, exercise therapy: daily, recommended clinic type: orthopedic clinic, estimated effect: medium”. The system passes the output of the generation unit to the selection unit, which generates a list of candidate clinics suitable for the treatment plan (e.g., a scored list considering geographic distance, specialty, past patient ratings, congestion status, etc.). The selection unit uses ranking learning algorithms or recommendation systems (e.g., collaborative filtering, content-based recommendation) to present the optimal clinic to the patient. The patient can select from the presented clinic list and make reservations or inquiries. The feedback unit collects the effects and impressions input by the patient after treatment (e.g., pain change score, treatment satisfaction, free-text comments, etc.) and re-inputs them to the AI model to continuously improve the personalization accuracy of the treatment plan. The AI model updates its weights through online learning or reinforcement learning using the feedback data as teacher signals, thereby improving the accuracy of subsequent treatment plan generation and clinic recommendation. Thus, the system achieves not only automation of human tasks but also technical improvements beyond conventional rule-based or manual judgment through high-dimensional feature extraction, nonlinear optimization, and real-time adaptive processing by AI (e.g., individualized optimization of treatment plans, improved accuracy of clinic selection, maximization of patient satisfaction, data-driven prediction of treatment effects, optimization of communication and computational load, etc.). The application field is not limited to services for low back pain patients but can be expanded to other chronic disease management, rehabilitation support, telemedicine platforms, and personalized healthcare in general. As a technical effect, the present invention enables integrated analysis of diverse data such as symptoms, treatment history, and lifestyle by AI, and provides optimized treatment plans and clinic recommendations for each patient in real time, thereby improving treatment effects, efficient use of medical resources, and qualitative improvement of patient experience. Furthermore, the internal processing of the AI model is based on concrete algorithm design that eliminates black-box descriptions, such as error minimization by loss functions, optimization by gradient descent of weights, and improvement of generalization performance by data augmentation and regularization. With these configurations, the present invention satisfies the technical requirements under U.S. patent law and achieves substantial improvement in computer technology.

The service for low back pain patients according to the embodiment comprises a reception unit, a generation unit, a selection unit, and a feedback unit. The reception unit is configured to input the patient's symptoms and health information. The patient's symptoms and health information may include, for example, medical history, allergy information, current symptoms, and the like, but are not limited to such examples. The reception unit enables the patient to input detailed information such as the degree of low back pain, onset timing, past treatment history, and lifestyle. The generation unit analyzes the information input by the reception unit using AI and generates an optimal treatment plan. For example, the generation unit proposes a treatment plan centered on stretching and massage based on the patient's symptoms and health information. The generation unit can also generate a customized treatment plan by considering the patient's lifestyle and past treatment history. The selection unit selects a clinic based on the treatment plan generated by the generation unit. For example, the selection unit enables the patient to select the optimal clinic from nearby clinics based on the treatment plan proposed by the AI. The selection unit can also make more appropriate selections by referring to clinic ratings and reviews. The feedback unit provides feedback on the progress of treatment at the clinic selected by the selection unit. For example, the feedback unit enables the patient to provide feedback on the effect and impressions online after receiving treatment. This allows real-time confirmation of treatment effects and modification of the treatment plan as needed. Thus, the service for low back pain patients according to the embodiment can provide individualized treatment plans based on the patient's symptoms and health information, select the optimal clinic, and provide feedback on the progress of treatment. For example, the reception unit provides an interface for inputting the patient's symptoms and health information. The generation unit analyzes the patient's symptoms and health information using AI and generates an optimal treatment plan. The selection unit selects the optimal clinic based on the treatment plan generated by the generation unit. The feedback unit provides feedback on the progress of treatment at the clinic selected by the selection unit. Thus, the service for low back pain patients can provide individualized treatment plans based on the patient's symptoms and health information, select the optimal clinic, and provide feedback on the progress of treatment. Specifically, the system can implement each module-reception unit, generation unit, selection unit, and feedback unit—as independent processes or microservices on a computer. The reception unit preprocesses the symptoms and health information input by the patient (e.g., low back pain score, onset date, treatment history, lifestyle, allergy information, etc.) as multidimensional vectors, applying numerical conversion, categorization, text embedding, and so on. For example, the low back pain score is represented as a numerical value from 0 to 10, the onset date as a date type, treatment history as one-hot encoding, and lifestyle as categorical encoding or text embedding vectors. The generation unit uses these vectors as input and generates candidate treatment plans using AI models such as multilayer perceptrons, convolutional neural networks, or Transformer-based large language models. Examples of AI model inputs include low back pain score=8, onset date=2024-06-01, treatment history=“chiropractic, acupuncture”, lifestyle=“long hours of desk work”, and so on. The AI model outputs structured data such as treatment content labels (e.g., stretching, massage, physical therapy), recommended frequency (e.g., three times a week), recommended duration (e.g., two months), recommended clinic type (e.g., orthopedic clinic, chiropractic clinic), and estimated effect score (e.g., high, medium, low). The selection unit receives the output from the generation unit and generates a list of candidate clinics considering geographic distance, specialty, patient ratings, congestion status, and so on. The selection unit uses ranking learning algorithms or recommendation systems (collaborative filtering, content-based recommendation, etc.) to present the optimal clinic to the patient. The patient can select from the presented clinic list and make reservations or inquiries. The feedback unit collects the effects and impressions input by the patient after treatment (e.g., pain change score, treatment satisfaction, free-text comments, etc.) and re-inputs them to the AI model to continuously improve the personalization accuracy of the treatment plan. The AI model updates its weights through online learning or reinforcement learning using the feedback data as teacher signals, thereby improving the accuracy of subsequent treatment plan generation and clinic recommendation. With these configurations, the system achieves not only automation of human tasks but also technical improvements beyond conventional rule-based or manual judgment through high-dimensional feature extraction, nonlinear optimization, and real-time adaptive processing by AI (e.g., individualized optimization of treatment plans, improved accuracy of clinic selection, maximization of patient satisfaction, data-driven prediction of treatment effects, optimization of communication and computational load, etc.). The application field is not limited to services for low back pain patients but can be expanded to other chronic disease management, rehabilitation support, telemedicine platforms, and personalized healthcare in general. As a technical effect, the present invention enables integrated analysis of diverse data such as symptoms, treatment history, and lifestyle by AI, and provides optimized treatment plans and clinic recommendations for each patient in real time, thereby improving treatment effects, efficient use of medical resources, and qualitative improvement of patient experience. Furthermore, the internal processing of the AI model is based on concrete algorithm design that eliminates black-box descriptions, such as error minimization by loss functions, optimization by gradient descent of weights, and improvement of generalization performance by data augmentation and regularization. With these configurations, the present invention satisfies the technical requirements under U.S. patent law and achieves substantial improvement in computer technology.

The generation unit can generate an optimal treatment plan based on the patient's symptoms and health information. For example, the generation unit proposes a treatment plan centered on stretching and massage based on the patient's symptoms and health information. The generation unit can also generate a customized treatment plan by considering the patient's lifestyle and past treatment history. For example, if the cause of the patient's low back pain is muscle tension, the generation unit proposes a treatment plan centered on stretching and massage. The generation unit can also generate a customized treatment plan by considering the patient's lifestyle and past treatment history. Thus, the generation unit can generate an optimal treatment plan based on the patient's symptoms and health information. Some or all of the above-described processing in the generation unit may be performed using AI or without using AI. For example, the generation unit can generate a treatment plan using an AI model that takes the patient's symptoms and health information as input and outputs an optimal treatment plan. Specifically, the generation unit receives the symptoms and health information input by the patient at the reception unit (e.g., low back pain score, onset timing, past treatment history, lifestyle, allergy information, etc.) as multidimensional vectors. The generation unit preprocesses these vectors by numerical conversion (e.g., low back pain score as an integer from 0 to 10, onset timing converted to epoch seconds), categorization (e.g., treatment history as one-hot encoding), and text embedding (e.g., lifestyle vectorized by a pre-trained model such as BERT). The generation unit uses these preprocessed vectors as input and generates candidate treatment plans using AI models such as multilayer perceptrons, convolutional neural networks, or Transformer-based large language models. Examples of AI model inputs include low back pain score=7, onset date=2024-05-01, past treatment history=“acupuncture, chiropractic”, lifestyle=“mainly desk work”; and low back pain score=3, onset date=2023-12-15, past treatment history=“exercise therapy”, lifestyle=“sports enthusiast”. The generation unit outputs structured data from the AI model, such as treatment content labels (e.g., stretching, massage, physical therapy), recommended frequency (e.g., three times a week), recommended duration (e.g., two months), recommended clinic type (e.g., orthopedic clinic, chiropractic clinic), and estimated effect score (e.g., high, medium, low). For example, “stretching: three times a week, massage: twice a month, recommended clinic type: chiropractic clinic, estimated effect: high” or “physical therapy: once a week, exercise therapy: daily, recommended clinic type: orthopedic clinic, estimated effect: medium” may be output. The generation unit uses a loss function (e.g., cross-entropy loss or mean squared error) within the AI model to minimize error and optimize weights by gradient descent. Furthermore, data augmentation and regularization (e.g., dropout, L2 regularization) are applied to improve generalization performance. The output of the AI model is passed to the subsequent selection unit and used for clinic recommendation and presentation to the patient. Thus, the generation unit achieves high-dimensional feature extraction, nonlinear optimization, and real-time adaptive processing without relying on simple rule-based judgment or heuristics, resulting in technical effects such as individualized optimization of treatment plans, improved accuracy of treatment effect prediction, computational efficiency, and maximization of patient satisfaction. The application field is not limited to services for low back pain patients but can be expanded to chronic disease management, rehabilitation support, telemedicine platforms, and personalized healthcare in general.

The selection unit can select a clinic based on the generated treatment plan. For example, the selection unit enables the patient to select the optimal clinic from nearby clinics based on the treatment plan proposed by the AI. The selection unit can make more appropriate selections by referring to clinic ratings and reviews. For example, the selection unit enables the patient to select the optimal clinic from nearby clinics based on the treatment plan proposed by the AI. The selection unit can make more appropriate selections by referring to clinic ratings and reviews. Thus, the selection unit can select the optimal clinic based on the generated treatment plan. Some or all of the above-described processing in the selection unit may be performed using AI or without using AI. For example, the selection unit can select a clinic using an AI model that takes the generated treatment plan as input and outputs the optimal clinic. Specifically, the selection unit receives the structured data of the treatment plan from the generation unit (e.g., treatment content labels, recommended frequency, recommended duration, recommended clinic type, estimated effect score, etc.) as input. The selection unit obtains multidimensional information such as geographic distance, specialty, patient ratings, congestion status, and review scores from the clinic database and preprocesses these as numerical vectors or categorical vectors. The selection unit uses ranking learning algorithms (e.g., pairwise ranking, listwise ranking) or recommendation systems (e.g., collaborative filtering, content-based recommendation, graph neural networks, etc.) to calculate matching scores between the treatment plan and clinic information. Examples of AI model inputs include “treatment content=stretching, recommended clinic type=chiropractic clinic, patient address=Chuo-ku, Tokyo, past rating=4.5” or “treatment content=physical therapy, recommended clinic type=orthopedic clinic, patient address=Kita-ku, Osaka, congestion status=available”. The AI model outputs a list of candidate clinics (e.g., clinic ID, score, recommended reason, distance, rating, etc.), such as “Clinic A: score 0.92, distance 1.2 km, rating 4.7” or “Clinic B: score 0.85, distance 2.0 km, rating 4.5”. The selection unit uses a loss function (e.g., ranking loss, hinge loss) within the AI model to optimize clinic selection accuracy and updates weights by gradient descent. Furthermore, the patient's past selection history and feedback data are reflected in online learning to continuously improve recommendation accuracy. The output of the AI model is presented to the patient, who can select a clinic from the list and make reservations or inquiries. Thus, the selection unit achieves high-dimensional feature integration, nonlinear optimization, and real-time recommendation without relying on simple geographic search or review reference, resulting in technical effects such as improved accuracy of clinic selection, maximization of patient satisfaction, and efficient use of medical resources. The application field includes medical institution recommendation, telemedicine platforms, and personalized healthcare in general.

The feedback unit can share the progress of treatment in real time. For example, the feedback unit enables the patient to provide feedback on the effect and impressions online after receiving treatment. The feedback unit can confirm the effect of treatment in real time and modify the treatment plan as needed. For example, the feedback unit enables the patient to provide feedback on the effect and impressions online after receiving treatment. This allows real-time confirmation of treatment effects and modification of the treatment plan as needed. Thus, the feedback unit can share the progress of treatment in real time. Some or all of the above-described processing in the feedback unit may be performed using AI or without using AI. For example, the feedback unit can share the progress of treatment using an AI model that takes the progress of treatment as input and outputs feedback. Specifically, the feedback unit preprocesses the effects and impressions input by the patient after treatment (e.g., pain change score, treatment satisfaction, free-text comments, clinic rating, etc.) as multidimensional vectors or text embedding vectors. The feedback unit uses these data as input to an AI model (e.g., multilayer perceptron, time series analysis model, Transformer-based large language model, etc.) to perform evaluation of treatment effects, proposals for modification of treatment plans, generation of progress graphs, generation of feedback messages for patients, and so on. Examples of AI model inputs include “pain score=2→5, treatment satisfaction=3, comment=‘Pain increased after treatment’” or “pain score=7→2, treatment satisfaction=5, comment=‘Very effective’”. The AI model outputs structured data or natural language text such as treatment effect evaluation (e.g., improvement, worsening, no change), proposals for modification of treatment plans (e.g., increase frequency of stretching, recommend changing clinic), progress graph data, and feedback messages for patients (e.g., ‘Let's add exercise therapy next time’). The feedback unit uses a loss function (e.g., classification loss, regression loss) within the AI model to optimize feedback accuracy and updates weights by gradient descent. Furthermore, the feedback unit accumulates patient-specific history and clinic-specific effect data and continuously optimizes the model through online learning or reinforcement learning. The output of the AI model is re-input to the generation unit or selection unit to improve the accuracy of treatment plan generation and clinic recommendation. Thus, the feedback unit achieves high-dimensional data analysis, nonlinear optimization, and real-time adaptive processing without relying on simple questionnaire aggregation or manual evaluation, resulting in technical effects such as visualization of treatment effects, improved personalization accuracy of treatment plans, and qualitative improvement of patient experience. The application field includes medical feedback management, telemedicine platforms, and personalized healthcare in general.

The generation unit can customize the treatment plan based on the patient's lifestyle and past treatment history. For example, the generation unit can generate a customized treatment plan by considering the patient's lifestyle and past treatment history. The generation unit can generate a customized treatment plan by considering the patient's lifestyle and past treatment history. For example, the generation unit can generate a customized treatment plan by considering the patient's lifestyle and past treatment history. Thus, the generation unit can customize the treatment plan by considering the patient's lifestyle and past treatment history. Some or all of the above-described processing in the generation unit may be performed using AI or without using AI. For example, the generation unit can generate a treatment plan using an AI model that takes the patient's lifestyle and past treatment history as input and outputs a customized treatment plan. Specifically, the generation unit preprocesses the lifestyle input by the patient at the reception unit (e.g., exercise frequency, dietary content, sleep duration, smoking/drinking habits, etc.) and past treatment history (e.g., treatment method, clinic, treatment duration, effect, etc.) as multidimensional vectors or categorical vectors. The generation unit uses these vectors as input to an AI model (e.g., multilayer perceptron, convolutional neural network, Transformer-based large language model, etc.) to generate an optimized treatment plan for each patient. Examples of AI model inputs include “exercise frequency=once a week, diet=high fat, sleep=6 hours, past treatment=chiropractic, acupuncture” or “exercise frequency=daily, diet=well-balanced, sleep=8 hours, past treatment=exercise therapy”. The AI model outputs structured data such as treatment content labels (e.g., stretching, massage, physical therapy), recommended frequency, recommended duration, recommended clinic type, and estimated effect score, such as “stretching: three times a week, massage: twice a month, recommended clinic type: chiropractic clinic, estimated effect: high”. The generation unit uses a loss function (e.g., cross-entropy loss, mean squared error) within the AI model to optimize the accuracy of treatment plan generation and updates weights by gradient descent. Furthermore, the generation unit reflects patient feedback data through online learning or reinforcement learning to continuously improve the personalization accuracy of the treatment plan. The output of the AI model is presented to the patient, who can review, select, or modify the treatment plan as needed. Thus, the generation unit achieves high-dimensional feature extraction, nonlinear optimization, and real-time adaptive processing without relying on simple heuristics or rule-based judgment, resulting in technical effects such as individualized optimization of treatment plans, improved accuracy of treatment effect prediction, and maximization of patient satisfaction. The application field includes chronic disease management, rehabilitation support, and personalized healthcare in general.

The feedback unit can modify the treatment plan based on patient feedback. For example, the feedback unit enables the patient to provide feedback on the effect and impressions online after receiving treatment. The feedback unit can confirm the effect of treatment in real time and modify the treatment plan as needed. For example, the feedback unit enables the patient to provide feedback on the effect and impressions online after receiving treatment. This allows real-time confirmation of treatment effects and modification of the treatment plan as needed. Thus, the feedback unit can modify the treatment plan based on patient feedback. Some or all of the above-described processing in the feedback unit may be performed using AI or without using AI. For example, the feedback unit can modify the treatment plan using an AI model that takes patient feedback as input and outputs modifications to the treatment plan. Specifically, the feedback unit receives multidimensional data such as pain change score input by the patient after treatment (e.g., before treatment=6, after treatment=3), treatment satisfaction (e.g., five-point scale), and free-text comments (e.g., ‘Low back pain was alleviated after treatment’). The feedback unit preprocesses these data as numerical vectors or text embedding vectors and inputs them to the AI model. The AI model may be a multilayer perceptron, time series analysis model, Transformer-based large language model, etc. Examples of AI model inputs include “pain score=6→3, satisfaction=4, comment=‘Low back pain was alleviated after treatment’” or “pain score=5→7, satisfaction=2, comment=‘Pain increased after treatment’”. The AI model outputs structured data or natural language text such as proposals for modification of treatment plans (e.g., increase frequency of stretching, recommend changing clinic, add/delete treatment content), estimated effect evaluation (e.g., improvement, worsening, no change), and feedback messages for patients (e.g., ‘Let's add exercise therapy next time’). For example, “increase stretching frequency from twice a week to four times a week” or “recommend changing clinic from orthopedic to chiropractic” may be output. The feedback unit uses classification loss or regression loss as a loss function within the AI model to optimize feedback accuracy and updates weights by gradient descent. Furthermore, the feedback unit accumulates patient-specific history and clinic-specific effect data and continuously optimizes the model through online learning or reinforcement learning. The output of the AI model is re-input to the generation unit or selection unit to improve the accuracy of treatment plan generation and clinic recommendation. Thus, the feedback unit achieves high-dimensional data analysis, nonlinear optimization, and real-time adaptive processing without relying on simple questionnaire aggregation or manual evaluation, resulting in technical effects such as visualization of treatment effects, improved personalization accuracy of treatment plans, and qualitative improvement of patient experience. The application field includes medical feedback management, telemedicine platforms, and personalized healthcare in general. As a technical effect, the present invention enables real-time analysis of patient feedback and automatic modification of treatment plans by AI, thereby maximizing treatment effects, efficient use of medical resources, and improving patient satisfaction. Furthermore, the internal processing of the AI model is based on concrete algorithm design that eliminates black-box descriptions, such as error minimization by loss functions, optimization by gradient descent of weights, and improvement of generalization performance by data augmentation and regularization. With these configurations, the present invention satisfies the technical requirements under U.S. patent law and achieves substantial improvement in computer technology.

The reception unit can estimate the patient's emotions and adjust the method of inputting symptoms or health information based on the estimated emotions. For example, if the patient is feeling stressed, the reception unit provides a simple interface and minimizes the input steps. If the patient is relaxed, the reception unit provides detailed input options and can propose customizable input methods. If the patient is in a hurry, the reception unit prioritizes voice input to enable rapid input of symptoms or health information. Thus, the reception unit can adjust the method of inputting symptoms or health information according to the patient's emotions. Emotion estimation is realized, for example, by using an emotion engine or generative AI with emotion estimation functions. Generative AI may be a text generation AI (e.g., LLM) or multimodal generative AI, but is not limited to such examples. Some or all of the above-described processing in the reception unit may be performed using AI or without using AI. For example, the reception unit can adjust the input method using an AI model that takes the patient's emotion data as input and outputs adjustments to the input method. Specifically, the reception unit preprocesses text data input by the patient (e.g., free description of symptoms, health information in selectable format, voice input data, facial image data, etc.) and operation logs during input (e.g., input speed, number of input interruptions, selection history of input interfaces, etc.) as multidimensional vectors or time-series tensors. The reception unit uses these data as input to an emotion estimation AI model (e.g., BERT-based emotion classification model, convolutional neural network for voice emotion recognition, multimodal Transformer, etc.) to estimate the patient's emotional state (e.g., stress, relaxation, haste, anxiety, etc.). Examples of AI model inputs include (1) “input text: ‘Recently, my back hurts and I can't sleep at night’, input speed: slow, facial image: frown”; and (2) “voice input: bright tone, input interface: voice prioritized, number of input interruptions: 0”. The AI model outputs structured data such as emotion labels (e.g., stress=high, relaxation=medium, haste=low), emotion scores (e.g., stress level 0.85, relaxation level 0.15), and recommended input mode (e.g., simple UI, detailed UI, voice input prioritized, etc.). For example, “stress=high, recommended input mode=simple UI” or “relaxation=high, recommended input mode=detailed UI” may be output. Based on the output of the AI model, the reception unit adjusts the number of display items in the interface, simplifies input steps, automatically activates voice input, and displays input assistance messages in real time. Furthermore, the patient's emotion estimation results are linked to subsequent generation units and feedback units and are also used for personalization in treatment plan generation and feedback display. Internal processing of the AI model includes optimization of emotion estimation accuracy by loss functions (e.g., cross-entropy loss), learning by gradient descent of weights, improvement of generalization performance by data augmentation (e.g., voice pitch conversion, text paraphrase generation) and regularization (e.g., dropout). Thus, the reception unit achieves high-dimensional feature extraction, nonlinear optimization, and real-time adaptive processing without relying on simple human observation or heuristics, providing the optimal input experience for each patient and resulting in technical effects such as improved input accuracy, reduced input burden, and maximization of patient satisfaction. The application field includes not only medical reception systems but also telemedicine platforms, personalized healthcare, customer support reception, and adaptive input support in the education field.

The reception unit can analyze the patient's past symptom input history and propose an optimal input method. For example, the reception unit automatically displays symptoms or health information that the patient has frequently input in the past as candidates. The reception unit can also preferentially propose input methods (voice, text, etc.) that the patient has used in the past. The reception unit can predict and propose symptoms or health information used at specific times based on the patient's past input history. Thus, the reception unit can analyze the patient's past symptom input history and propose an optimal input method. Some or all of the above-described processing in the reception unit may be performed using AI or without using AI. For example, the reception unit can propose an input method using an AI model that takes the patient's past input history as input and outputs the optimal input method. Specifically, the reception unit manages the patient's accumulated past symptom input history data (e.g., input date and time, input content, input method, input time required, selection history of input interface during input, etc.) as a time-series database. The reception unit preprocesses these history data as time-series vectors or categorical vectors and inputs them to an AI model (e.g., LSTM for time-series analysis, multilayer perceptron for input pattern classification, Transformer for recommendation systems, etc.). Examples of AI model inputs include (1) “input methods in the past month: voice input=70%, text input=30%, input content: low back pain, stiff shoulders, sleep disorders, input time: mostly at night”; and (2) “past input content: low back pain score=5-7, input method: text, average input time: 2 minutes”. The AI model outputs structured data such as recommended input method (e.g., voice input prioritized, text input prioritized, hybrid input), candidate item list (e.g., auto-completion candidates for frequently occurring symptoms), and customization settings for input interface (e.g., dark mode at night, simple mode in the morning). For example, “recommended input method: voice input, candidate items: low back pain, stiff shoulders, interface: night mode” may be output. Based on the output of the AI model, the reception unit applies auto-completion of frequently occurring items, automatic switching of input methods, and UI optimization according to time of day to the patient's input screen in real time. Furthermore, the patient's input history is used for online learning or reinforcement learning to update the AI model's weights, continuously improving personalization accuracy. Internal processing of the AI model includes optimization by loss functions (e.g., cross-entropy loss, time-series prediction loss), learning by gradient descent of weights, and improvement of generalization performance by data augmentation (e.g., synthesis of input patterns) and regularization (e.g., dropout). Thus, the reception unit achieves high-dimensional feature extraction, nonlinear optimization, and real-time adaptive processing without relying on simple history reference or heuristics, providing the optimal input experience for each patient and resulting in technical effects such as improved input efficiency, reduced input errors, and maximization of patient satisfaction. The application field includes not only medical reception systems but also telemedicine platforms, personalized healthcare, customer support reception, and adaptive input support in the education field.

The reception unit can customize input items based on the patient's current living conditions and areas of interest when inputting symptoms or health information. For example, if the patient is interested in exercise, the reception unit prioritizes the display of input items related to exercise. If the patient is interested in diet, the reception unit can also prioritize the display of input items related to diet. If the patient is interested in stress management, the reception unit can also prioritize the display of input items related to stress management. Thus, the reception unit can customize input items based on the patient's current living conditions and areas of interest. Some or all of the above-described processing in the reception unit may be performed using AI or without using AI. For example, the reception unit can customize input items using an AI model that takes the patient's living conditions and areas of interest as input and outputs customization of input items. Specifically, the reception unit preprocesses living condition data input by the patient (e.g., exercise frequency, dietary content, sleep duration, stress level, hobbies/areas of interest, etc.), past input history, questionnaire responses, activity data from wearable devices, etc., as multidimensional vectors or categorical vectors. The reception unit uses these data as input to an AI model for customizing input items (e.g., multilayer perceptron, content-based recommendation model, multimodal Transformer, etc.) to generate an optimal input item list for each patient. Examples of AI model inputs include (1) “exercise frequency=three times a week, areas of interest=strength training, yoga, sleep=7 hours”; and (2) “diet=high protein, low fat, areas of interest=diet management, nutrition”. The AI model outputs structured data such as prioritized input item list (e.g., exercise habits, dietary content, stress management, sleep status), order of input items, and display flags for input items. For example, “priority items: exercise habits, stress management, sleep”, “display order: exercise→diet→stress” may be output. Based on the output of the AI model, the reception unit optimizes the order and content of items displayed on the patient's input screen in real time, providing an input experience tailored to the patient's interests. Furthermore, the patient's areas of interest and living conditions are used for online learning or reinforcement learning to update the AI model's weights, continuously improving personalization accuracy. Internal processing of the AI model includes optimization by loss functions (e.g., ranking loss, cross-entropy loss), learning by gradient descent of weights, and improvement of generalization performance by data augmentation (e.g., clustering of areas of interest) and regularization (e.g., dropout). Thus, the reception unit achieves high-dimensional feature extraction, nonlinear optimization, and real-time adaptive processing without relying on simple item selection or heuristics, providing the optimal input experience for each patient and resulting in technical effects such as improved input efficiency, improved input accuracy, and maximization of patient satisfaction. The application field includes not only medical reception systems but also telemedicine platforms, personalized healthcare, customer support reception, and adaptive input support in the education field.

The reception unit can estimate the patient's emotions and determine the priority of information to be input based on the estimated emotions. For example, if the patient is feeling stressed, the reception unit prompts the patient to input important information first. If the patient is relaxed, the reception unit can also prompt the patient to input detailed information. If the patient is in a hurry, the reception unit can also prompt the patient to input the most important information first. Thus, the reception unit can determine the priority of information to be input according to the patient's emotions. Emotion estimation is realized, for example, by using an emotion engine or generative AI with emotion estimation functions. Generative AI may be a text generation AI (e.g., LLM) or multimodal generative AI, but is not limited to such examples. Some or all of the above-described processing in the reception unit may be performed using AI or without using AI. For example, the reception unit can determine the priority of information using an AI model that takes the patient's emotion data as input and determines the priority of information. Specifically, the reception unit preprocesses text data input by the patient (e.g., free description of symptoms, health information in selectable format, voice input data, facial image data, etc.) and operation logs during input (e.g., input speed, number of input interruptions, selection history of input interfaces, etc.) as multidimensional vectors or time-series tensors. The reception unit uses these data as input to an emotion estimation AI model (e.g., BERT-based emotion classification model, convolutional neural network for voice emotion recognition, multimodal Transformer, etc.) to estimate the patient's emotional state (e.g., stress, relaxation, haste, anxiety, etc.). Examples of AI model inputs include “input text: ‘Recently, my back hurts and I can't sleep at night’, input speed: slow, facial image: frown” or “voice input: bright tone, input interface: voice prioritized, number of input interruptions: 0”. The AI model outputs structured data such as emotion labels (e.g., stress=high, relaxation=medium, haste=low), emotion scores (e.g., stress level 0.85, relaxation level 0.15), and recommended input mode (e.g., simple UI, detailed UI, voice input prioritized, etc.). For example, “stress=high, recommended input mode=simple UI” or “relaxation=high, recommended input mode=detailed UI” may be output. Based on the output of the AI model, the reception unit adjusts the priority of information items displayed on the input screen in real time. For example, if the stress level is high, only the most important items such as “current pain level” and “onset timing” are displayed first, and detailed lifestyle and past treatment history are postponed. Conversely, if the relaxation level is high, the reception unit actively prompts input of detailed health information, lifestyle, allergy information, etc. If the patient is in a hurry, voice input and one-tap options are prioritized to minimize input burden. Internal processing of the AI model includes optimization of emotion estimation accuracy by loss functions (e.g., cross-entropy loss), learning by gradient descent of weights, improvement of generalization performance by data augmentation (e.g., voice pitch conversion, text paraphrase generation) and regularization (e.g., dropout). Furthermore, the patient's emotion estimation history and input behavior data are used for online learning or reinforcement learning to update the model's weights, continuously improving personalization accuracy. The output of the AI model is also linked to subsequent generation units and feedback units and used for personalization in treatment plan generation and feedback display. Thus, the reception unit achieves high-dimensional feature extraction, nonlinear optimization, and real-time adaptive processing without relying on simple human observation or heuristics, providing the optimal input experience and information presentation order for each patient and resulting in technical effects such as improved input accuracy, reduced input burden, and maximization of patient satisfaction. The application field includes not only medical reception systems but also telemedicine platforms, personalized healthcare, customer support reception, and adaptive input support in the education field.

The reception unit can prioritize the input of highly relevant information by considering the patient's geographic location when inputting symptoms or health information. For example, if the patient lives in a specific region, the reception unit prioritizes the input of information related to diseases prevalent in that region. If the patient is traveling, the reception unit can also prioritize the input of information related to health risks at the travel destination. If the patient lives in a specific climate, the reception unit can also prioritize the input of health information related to that climate. Thus, the reception unit can prioritize the input of highly relevant information by considering the patient's geographic location. Some or all of the above-described processing in the reception unit may be performed using AI or without using AI. For example, the reception unit can input information using an AI model that takes the patient's geographic location as input and prioritizes the input of highly relevant information. Specifically, the reception unit preprocesses the patient's current location information (e.g., GPS coordinates, address, region estimated from IP address, etc.), past movement history, travel plans, climate data (e.g., temperature, humidity, pollen count, etc.) as multidimensional vectors or categorical vectors. The reception unit uses these data as input to a geographic information-linked AI model (e.g., multilayer perceptron with geographic clustering, content recommendation model linked to climate data, multimodal Transformer, etc.) to generate a list of health information items to be prioritized for input for each patient. Examples of AI model inputs include “current location=Chuo-ku, Tokyo, season=spring, pollen count=high” or “current location=Naha City, Okinawa Prefecture, temperature=30° C., traveling=yes”. The AI model outputs structured data such as prioritized input item list (e.g., pollen allergy symptoms, heat stroke risk, infectious disease prevalence information), order of input items, and display flags for input items. For example, “priority items: pollen allergy symptoms, allergy information, respiratory symptoms”, “display order: infectious disease→climate-related→general symptoms” may be output. Based on the output of the AI model, the reception unit optimizes the order and content of items displayed on the patient's input screen in real time, providing an input experience tailored to regional health risks and prevalence. Furthermore, the patient's geographic location and climate data are used for online learning or reinforcement learning to update the AI model's weights, continuously improving personalization accuracy. Internal processing of the AI model includes optimization by loss functions (e.g., ranking loss, cross-entropy loss), learning by gradient descent of weights, and improvement of generalization performance by data augmentation (e.g., synthesis of symptom frequency data by region) and regularization (e.g., dropout). Thus, the reception unit achieves high-dimensional feature extraction, nonlinear optimization, and real-time adaptive processing without relying on simple regional reference or heuristics, providing the optimal input experience for each patient and resulting in technical effects such as improved input efficiency, improved input accuracy, and maximization of patient satisfaction. The application field includes not only medical reception systems but also telemedicine platforms, personalized healthcare, health management for travelers, and health information input support during disasters.

The reception unit can analyze the patient's social media activity and input relevant information when inputting symptoms or health information. For example, the reception unit proposes relevant input items based on health information shared by the patient on social media. The reception unit can also obtain information from health-related accounts followed by the patient on social media and propose input items. The reception unit can also obtain information from health-related groups the patient participates in on social media and propose input items. Thus, the reception unit can analyze the patient's social media activity and input relevant information. Some or all of the above-described processing in the reception unit may be performed using AI or without using AI. For example, the reception unit can input information using an AI model that takes the patient's social media activity as input and inputs relevant information. Specifically, the reception unit preprocesses social media post data obtained within the scope permitted by the patient (e.g., post text about health status, images, videos, hashtags, check-in history, etc.), list of followed accounts, group participation information, like history, etc., as multidimensional vectors or text embedding vectors. The reception unit uses these data as input to a social media analysis AI model (e.g., BERT-based text classification model, CNN for image feature extraction, multimodal Transformer, etc.) to generate a list of highly relevant health information items or candidate input items for each patient. Examples of AI model inputs include “post text: ‘Recently, my low back pain has worsened’, followed accounts: health information providers, participating groups: low back pain countermeasure community” or “image post: photo during exercise, hashtag: #lowbackpainimprovement”. The AI model outputs structured data such as prioritized input item list (e.g., low back pain score, exercise habits, treatment history), candidate input content (e.g., symptoms or treatments extracted from past posts), and customization settings for input interface (e.g., auto-completion of frequently used items). For example, “priority items: low back pain score, exercise frequency, treatment history”, “candidate content: recent exercise content, medications taken” may be output. Based on the output of the AI model, the reception unit applies auto-completion of past post content, prioritized display of related items, and addition of question items based on group participation history to the patient's input screen in real time. Furthermore, the patient's social media activity data are used for online learning or reinforcement learning to update the AI model's weights, continuously improving personalization accuracy. Internal processing of the AI model includes optimization by loss functions (e.g., cross-entropy loss, text classification loss), learning by gradient descent of weights, and improvement of generalization performance by data augmentation (e.g., paraphrase generation of post text, image rotation/enlargement) and regularization (e.g., dropout). Thus, the reception unit achieves high-dimensional feature extraction, nonlinear optimization, and real-time adaptive processing without relying on simple post reference or heuristics, providing the optimal input experience for each patient and resulting in technical effects such as improved input efficiency, improved input accuracy, and maximization of patient satisfaction. The application field includes not only medical reception systems but also telemedicine platforms, personalized healthcare, health management apps, and customer support reception.

The generation unit can estimate the patient's emotions and adjust the method of expressing the treatment plan based on the estimated emotions. For example, if the patient is relaxed, the generation unit provides a treatment plan including detailed explanations. If the patient is feeling stressed, the generation unit can also provide a simple and concise treatment plan. If the patient is in a hurry, the generation unit can also provide a treatment plan that can be quickly understood. Thus, the generation unit can adjust the method of expressing the treatment plan according to the patient's emotions. Emotion estimation is realized, for example, by using an emotion engine or generative AI with emotion estimation functions. Generative AI may be a text generation AI (e.g., LLM) or multimodal generative AI, but is not limited to such examples. Some or all of the above-described processing in the generation unit may be performed using AI or without using AI. For example, the generation unit can adjust the method of expression using an AI model that takes the patient's emotion data as input and adjusts the method of expressing the treatment plan. Specifically, the generation unit preprocesses emotion data received from the reception unit or feedback unit (e.g., stress level, relaxation level, degree of haste, etc. as scored numerical vectors, emotion labels, emotion estimation time-series data, etc.) as multidimensional vectors. The generation unit integrates these emotion data with vectors of the patient's symptoms, health information, and treatment history, and inputs them to a treatment plan generation AI model (e.g., Transformer-based large language model, conditional generation network, multimodal neural network, etc.). Examples of AI model inputs include (1) “emotion: stress level 0.85, relaxation level 0.10, haste level 0.05, symptoms: low back pain score 7, onset date 2024-05-01, treatment history: acupuncture, chiropractic”; and (2) “emotion: relaxation level 0.90, stress level 0.05, symptoms: low back pain score 3, onset date 2023-12-15, treatment history: exercise therapy”. The AI model outputs parameters for controlling the method of expressing the treatment plan (e.g., explanation detail level=high, medium, low; summary rate; natural language generation template selection flag, etc.) and the actual treatment plan text (e.g., “stretching: three times a week, massage: twice a month, recommended reason: expected to relieve muscle tension” or “a plan centered on stretching and massage that allows you to feel the effect in a short period” etc.). The AI model uses loss functions (e.g., explanation detail level classification loss, natural language generation loss) internally to optimize the accuracy of generating the method of expression according to the patient's emotional state by optimizing weights through gradient descent. Furthermore, feedback from the patient (e.g., evaluation of the understandability of the treatment plan, satisfaction with the amount of explanation, etc.) is reflected in online learning or reinforcement learning to continuously improve the personalization accuracy of the model. The output of the AI model is displayed in real time on the patient's device or web interface, allowing the patient to receive the optimal treatment plan explanation according to their emotional state. As a technical effect, the generation unit achieves high-dimensional emotion feature extraction, nonlinear optimization, and real-time adaptive processing without relying on simple template switching or heuristics, providing the optimal method of expressing the treatment plan for each patient and resulting in improved understanding of the treatment plan, reduced explanation burden, and maximization of patient satisfaction. The application field includes medical explanation text generation, personalized healthcare, telemedicine platforms, and adaptive teaching material generation in the education field. Furthermore, the internal processing of the AI model is based on concrete algorithm design that eliminates black-box descriptions, such as error minimization by loss functions, optimization by gradient descent of weights, and improvement of generalization performance by data augmentation and regularization, thereby achieving improvements in computer technology itself (e.g., computational efficiency of explanation generation, automatic optimization of explanation quality, reduction of communication load, etc.).

The generation unit can adjust the level of detail of the treatment plan based on the importance of the patient's symptoms when generating the treatment plan. For example, if the patient's symptoms are severe, the generation unit provides a detailed treatment plan. If the patient's symptoms are mild, the generation unit can also provide a concise treatment plan. If the patient's symptoms are moderate, the generation unit can also provide a treatment plan with an appropriate level of detail. Thus, the generation unit can adjust the level of detail of the treatment plan based on the importance of the patient's symptoms. Some or all of the above-described processing in the generation unit may be performed using AI or without using AI. For example, the generation unit can adjust the level of detail using an AI model that takes the importance of the patient's symptoms as input and adjusts the level of detail of the treatment plan. Specifically, the generation unit preprocesses symptom data received from the reception unit (e.g., low back pain score, duration of pain, impact on daily life, risk of complications, etc.) as multidimensional vectors and scores the importance of symptoms (e.g., severe=0.9, moderate=0.5, mild=0.2, etc.). The generation unit integrates these symptom importance vectors with the patient's health information and treatment history and inputs them to a treatment plan detail level control AI model (e.g., conditional generation network, Transformer-based large language model, etc.). Examples of AI model inputs include (1) “symptom score: severe=0.9, low back pain score=9, onset date=2024-05-01, treatment history: acupuncture”; and (2) “symptom score: mild=0.2, low back pain score=3, onset date=2023-12-15, treatment history: exercise therapy”. The AI model outputs parameters for the level of detail of the treatment plan (e.g., detail level=high, medium, low; length of explanation text; number of recommended items, etc.) and the actual treatment plan text (e.g., “stretching: three times a week, massage: twice a month, recommended reason: expected to relieve muscle tension” or “a plan centered on stretching and massage that allows you to feel the effect in a short period” etc.). The AI model uses loss functions (e.g., detail level classification loss, natural language generation loss) internally to optimize the accuracy of generating the level of detail of the treatment plan according to the importance of symptoms by optimizing weights through gradient descent. Furthermore, feedback from the patient (e.g., evaluation of the understandability of the treatment plan, satisfaction with the amount of explanation, etc.) is reflected in online learning or reinforcement learning to continuously improve the personalization accuracy of the model. The output of the AI model is displayed in real time on the patient's device or web interface, allowing the patient to receive the optimal treatment plan explanation according to the importance of their symptoms. As a technical effect, the generation unit achieves high-dimensional symptom feature extraction, nonlinear optimization, and real-time adaptive processing without relying on simple template switching or heuristics, providing the optimal level of detail of the treatment plan for each patient and resulting in improved understanding of the treatment plan, reduced explanation burden, and maximization of patient satisfaction. The application field includes medical explanation text generation, personalized healthcare, telemedicine platforms, and adaptive teaching material generation in the education field. Furthermore, the internal processing of the AI model is based on concrete algorithm design that eliminates black-box descriptions, such as error minimization by loss functions, optimization by gradient descent of weights, and improvement of generalization performance by data augmentation and regularization, thereby achieving improvements in computer technology itself (e.g., computational efficiency of explanation generation, automatic optimization of explanation quality, reduction of communication load, etc.).

The generation unit can apply different treatment algorithms according to the category of the patient's symptoms when generating the treatment plan. For example, in the case of low back pain due to muscle tension, the generation unit provides a treatment plan centered on stretching and massage. In the case of low back pain due to herniated disc, the generation unit can also provide a treatment plan including physical therapy or surgery. In the case of chronic low back pain, the generation unit can also provide a treatment plan including long-term rehabilitation. Thus, the generation unit can apply different treatment algorithms according to the category of the patient's symptoms. Some or all of the above-described processing in the generation unit may be performed using AI or without using AI. For example, the generation unit can generate a treatment plan using an AI model that takes the category of the patient's symptoms as input and applies different treatment algorithms. Specifically, the generation unit preprocesses symptom data received from the reception unit (e.g., symptom category label=muscular, disc-related, chronic, etc.; symptom feature vector; image diagnosis result vector, etc.) as multidimensional vectors and estimates the symptom category using a symptom category classification AI model (e.g., convolutional neural network for image classification, BERT-based text classification, multimodal fusion model, etc.). The generation unit uses a treatment algorithm selection module (e.g., rule-based branching, conditional generation network, algorithm selection neural network, etc.) according to the estimated symptom category to select the optimal treatment plan generation AI model (e.g., model for muscular low back pain, model for disc-related low back pain, model for chronic low back pain, etc.) and generate the treatment plan. Examples of AI model inputs include (1) “symptom category: muscular, low back pain score=7, onset date=2024-05-01, treatment history: acupuncture, chiropractic”; and (2) “symptom category: disc-related, image diagnosis: herniation observed, treatment history: physical therapy”. The AI model outputs structured data such as treatment content labels (e.g., stretching, massage, physical therapy, surgery), recommended frequency, recommended duration, recommended clinic type, and estimated effect score, such as “stretching: three times a week, massage: twice a month, recommended clinic type: chiropractic clinic, estimated effect: high” or “physical therapy: once a week, surgery considered, recommended clinic type: orthopedic clinic, estimated effect: medium”. The AI model uses loss functions (e.g., cross-entropy loss, treatment effect prediction loss) internally to optimize the accuracy of treatment plan generation for each symptom category and updates weights by gradient descent. Furthermore, patient feedback data (e.g., treatment effect, satisfaction, etc.) are reflected in online learning or reinforcement learning to continuously improve the personalization accuracy of the model. The output of the AI model is displayed in real time on the patient's device or web interface, allowing the patient to receive the optimal treatment plan for their symptom category. As a technical effect, the generation unit achieves high-dimensional symptom feature extraction, nonlinear optimization, and real-time adaptive processing without relying on simple rule-based branching or heuristics, providing optimized treatment plan generation for each symptom category and resulting in improved treatment effects, reduced explanation burden, and maximization of patient satisfaction. The application field includes medical explanation text generation, personalized healthcare, telemedicine platforms, and disease-specific treatment support systems. Furthermore, the internal processing of the AI model is based on concrete algorithm design that eliminates black-box descriptions, such as error minimization by loss functions, optimization by gradient descent of weights, and improvement of generalization performance by data augmentation and regularization, thereby achieving improvements in computer technology itself (e.g., automation of treatment algorithm selection, computational efficiency, automatic optimization of explanation quality, etc.).

The generation unit can estimate the patient's emotions and adjust the length of the treatment plan based on the estimated emotions. For example, if the patient is relaxed, the generation unit provides a longer treatment plan including detailed explanations. If the patient is feeling stressed, the generation unit can also provide a short and concise treatment plan. If the patient is in a hurry, the generation unit can also provide a short treatment plan that can be quickly understood. Thus, the generation unit can adjust the length of the treatment plan according to the patient's emotions. Emotion estimation is realized, for example, by using an emotion engine or generative AI with emotion estimation functions. Generative AI may be a text generation AI (e.g., LLM) or multimodal generative AI, but is not limited to such examples. Some or all of the above-described processing in the generation unit may be performed using AI or without using AI. For example, the generation unit can adjust the length of the treatment plan using an AI model that takes the patient's emotion data as input and adjusts the length of the treatment plan. Specifically, the generation unit preprocesses emotion data received from the reception unit or feedback unit (e.g., stress level, relaxation level, degree of haste, etc. as scored numerical vectors, emotion labels, emotion estimation time-series data, etc.) as multidimensional vectors. The generation unit integrates these emotion data with vectors of the patient's symptoms, health information, and treatment history, and inputs them to a treatment plan length control AI model (e.g., Transformer-based large language model, conditional generation network, multimodal neural network, etc.). Examples of AI model inputs include (1) “emotion: stress level 0.85, relaxation level 0.10, haste level 0.05, symptoms: low back pain score 7, onset date 2024-05-01, treatment history: acupuncture, chiropractic”; and (2) “emotion: relaxation level 0.90, stress level 0.05, symptoms: low back pain score 3, onset date 2023-12-15, treatment history: exercise therapy”. The AI model outputs parameters for the length of the treatment plan (e.g., length=long, medium, short; length of explanation text; number of recommended items, etc.) and the actual treatment plan text (e.g., “stretching: three times a week, massage: twice a month, recommended reason: expected to relieve muscle tension” or “a plan centered on stretching and massage that allows you to feel the effect in a short period” etc.). The AI model uses loss functions (e.g., length classification loss, natural language generation loss) internally to optimize the accuracy of generating the length of the treatment plan according to the patient's emotional state by optimizing weights through gradient descent. Furthermore, feedback from the patient (e.g., evaluation of the understandability of the treatment plan, satisfaction with the amount of explanation, etc.) is reflected in online learning or reinforcement learning to continuously improve the personalization accuracy of the model. The output of the AI model is displayed in real time on the patient's device or web interface, allowing the patient to receive the optimal treatment plan explanation according to their emotional state. As a technical effect, the generation unit achieves high-dimensional emotion feature extraction, nonlinear optimization, and real-time adaptive processing without relying on simple template switching or heuristics, providing the optimal length of the treatment plan for each patient and resulting in improved understanding of the treatment plan, reduced explanation burden, and maximization of patient satisfaction. The application field includes medical explanation text generation, personalized healthcare, telemedicine platforms, and adaptive teaching material generation in the education field. Furthermore, the internal processing of the AI model is based on concrete algorithm design that eliminates black-box descriptions, such as error minimization by loss functions, optimization by gradient descent of weights, and improvement of generalization performance by data augmentation and regularization, thereby achieving improvements in computer technology itself (e.g., computational efficiency of explanation generation, automatic optimization of explanation quality, reduction of communication load, etc.).

The generation unit can determine the priority of the treatment plan based on the onset timing of the patient's symptoms when generating the treatment plan. For example, for recently developed symptoms, the generation unit provides a treatment plan that prioritizes prompt treatment. For symptoms that have persisted for a long time, the generation unit can also provide a treatment plan that prioritizes long-term treatment. For recurrent symptoms, the generation unit can also provide a treatment plan that emphasizes recurrence prevention. Thus, the generation unit can determine the priority of the treatment plan based on the onset timing of the patient's symptoms. Some or all of the above-described processing in the generation unit may be performed using AI or without using AI. For example, the generation unit can generate a treatment plan using an AI model that takes the onset timing of the patient's symptoms as input and determines the priority of the treatment plan. Specifically, the generation unit preprocesses symptom data received from the reception unit (e.g., onset timing, duration of symptoms, number of recurrences, past treatment history, etc.) as multidimensional vectors, converts onset timing to epoch seconds or days, and scores the novelty, chronicity, and recurrence of symptoms (e.g., new=0.9, chronic=0.7, recurrent=0.8, etc.). The generation unit integrates these onset timing vectors with the patient's health information and treatment history and inputs them to a treatment plan priority determination AI model (e.g., conditional generation network, Transformer-based large language model, etc.). Examples of AI model inputs include (1) “onset timing=2024-06-01, duration=3 days, novelty=high, treatment history=none”; and (2) “onset timing=2023-01-01, duration=1 year, chronicity=high, treatment history=multiple times”. The AI model outputs parameters for the priority of the treatment plan (e.g., priority=high, medium, low; recommended treatment start timing; treatment duration, etc.) and the actual treatment plan text (e.g., “Prompt treatment is recommended for acute symptoms”, “Long-term rehabilitation is recommended for chronic symptoms”, “A plan emphasizing lifestyle improvement for recurrence prevention” etc.). The AI model uses loss functions (e.g., priority classification loss, natural language generation loss) internally to optimize the accuracy of generating the priority of the treatment plan according to onset timing by optimizing weights through gradient descent. Furthermore, feedback from the patient (e.g., satisfaction with treatment start timing, evaluation of treatment duration, etc.) is reflected in online learning or reinforcement learning to continuously improve the personalization accuracy of the model. The output of the AI model is displayed in real time on the patient's device or web interface, allowing the patient to receive the optimal treatment plan according to the onset timing of their symptoms. As a technical effect, the generation unit achieves high-dimensional time-series feature extraction, nonlinear optimization, and real-time adaptive processing without relying on simple heuristics or rule-based judgment, providing the optimal priority of the treatment plan for each patient and resulting in improved timeliness of treatment initiation, maximized treatment effects, and improved patient satisfaction. The application field includes medical explanation text generation, personalized healthcare, telemedicine platforms, and chronic disease management. Furthermore, the internal processing of the AI model is based on concrete algorithm design that eliminates black-box descriptions, such as error minimization by loss functions, optimization by gradient descent of weights, and improvement of generalization performance by data augmentation and regularization, thereby achieving improvements in computer technology itself (e.g., computational efficiency of priority determination, automatic optimization of explanation quality, reduction of communication load, etc.).

The generation unit can adjust the order of the treatment plan based on the relevance of the patient's symptoms when generating the treatment plan. For example, if multiple symptoms are related, the generation unit provides a treatment plan that starts with the most important symptom. If the symptoms are independent, the generation unit can also provide individual treatment plans for each symptom. If the symptoms affect each other, the generation unit can also provide a treatment plan that takes mutual influence into account. Thus, the generation unit can adjust the order of the treatment plan based on the relevance of the patient's symptoms. Some or all of the above-described processing in the generation unit may be performed using AI or without using AI. For example, the generation unit can generate a treatment plan using an AI model that takes the relevance of the patient's symptoms as input and adjusts the order of the treatment plan. Specifically, the generation unit preprocesses multiple symptom data received from the reception unit (e.g., symptom labels, relevance scores between symptoms, symptom onset time series, risk of complications, etc.) as multidimensional vectors or graph-structured data. The generation unit uses these symptom relevance data as input to a symptom relevance analysis AI model (e.g., graph neural network, multitask learning model, Transformer-based large language model, etc.) to estimate the priority and order of treatment among symptoms. Examples of AI model inputs include (1) “Symptom A=low back pain, Symptom B=stiff shoulders, relevance=0.8, onset date A=2024-05-01, onset date B=2024-04-15”; and (2) “Symptom A=low back pain, Symptom B=sleep disorder, relevance=0.3, high independence”. The AI model outputs parameters for the order of the treatment plan (e.g., prioritized symptom list for treatment, simultaneous treatment recommendation flag, order of treatment initiation, etc.) and the actual treatment plan text (e.g., “First, prioritize treatment of low back pain, then treat stiff shoulders”, “Since low back pain and sleep disorder are independent, proceed with treatment in parallel” etc.). The AI model uses loss functions (e.g., order classification loss, relevance estimation loss, natural language generation loss) internally to optimize the accuracy of generating the order of the treatment plan according to symptom relevance by optimizing weights through gradient descent. Furthermore, feedback from the patient (e.g., degree of acceptance of treatment order, evaluation of treatment effects, etc.) is reflected in online learning or reinforcement learning to continuously improve the personalization accuracy of the model. The output of the AI model is displayed in real time on the patient's device or web interface, allowing the patient to receive the optimal order of the treatment plan according to the relevance of their symptoms. As a technical effect, the generation unit achieves high-dimensional symptom relevance extraction, nonlinear optimization, and real-time adaptive processing without relying on simple symptom listing or heuristics, providing the optimal order of the treatment plan for each patient and resulting in maximized treatment effects, improved treatment efficiency, and improved patient satisfaction. The application field includes medical explanation text generation, personalized healthcare, telemedicine platforms, and multiple disease management support. Furthermore, the internal processing of the AI model is based on concrete algorithm design that eliminates black-box descriptions, such as error minimization by loss functions, optimization by gradient descent of weights, and improvement of generalization performance by data augmentation and regularization, thereby achieving improvements in computer technology itself (e.g., computational efficiency of order determination, automatic optimization of explanation quality, reduction of communication load, etc.).

The selection unit can estimate the patient's emotions and adjust the selection criteria for clinics based on the estimated emotions. For example, when the patient is relaxed, the selection unit provides detailed information to support the selection of a clinic. When the patient is feeling stressed, the selection unit can provide simple information to enable quick selection of a clinic. When the patient is in a hurry, the selection unit can prioritize proposing the highest-rated clinics. Thus, the selection unit can adjust the selection criteria for clinics according to the patient's emotions. Emotion estimation is implemented, for example, by using an emotion estimation function with an emotion engine or generative AI. Generative AI may include text generation AI (e.g., LLM) or multimodal generative AI, but is not limited to these examples. Some or all of the above-described processing in the selection unit may be performed using AI or without using AI. For example, the selection unit can use the patient's emotion data as input and adjust the selection criteria using an AI model for clinic selection criteria adjustment. Specifically, the selection unit preprocesses the patient's emotion data (e.g., scores for stress level, relaxation level, urgency, emotion labels, emotion estimation time-series data, etc.) received from the reception unit or feedback unit as multidimensional vectors. The selection unit integrates these emotion data with vectors for the patient's symptoms, health information, treatment history, geographic information, and candidate clinic lists, and inputs them into an AI model for clinic selection criteria adjustment (e.g., Transformer-based large language model, conditional recommendation network, multimodal neural network, etc.). Examples of AI model inputs include (1) “Emotion: stress level 0.85, relaxation level 0.10, urgency 0.05; symptoms: low back pain score 7; onset date 2024-05-01; treatment history: acupuncture, chiropractic; number of candidate clinics=10” and (2) “Emotion: relaxation level 0.90, stress level 0.05; symptoms: low back pain score 3; onset date 2023-12-15; treatment history: exercise therapy; number of candidate clinics=5”. The selection unit generates, as outputs of the AI model, clinic selection criteria parameters (e.g., information detail level: high/medium/low, number of display items, priority display flag, recommendation algorithm selection flag, etc.) and actual candidate clinic lists (e.g., lists with detailed information, simple display lists, ranking lists, etc.). For example, outputs such as “detail level=high, display all candidates, with detailed information” or “detail level=low, display only top 3, ranked by evaluation” are generated. The selection unit optimizes in real time the display content, display order, and information amount of the candidate clinic list on the patient's device or web interface based on the AI model output. Furthermore, the patient's emotion estimation results and selection behavior history are used for online learning or reinforcement learning to update the AI model weights, continuously improving personalization accuracy. Internal processing of the AI model includes optimization of selection criteria adjustment accuracy using loss functions (e.g., cross-entropy loss, ranking loss), learning by gradient descent of weights, and implementation of generalization performance improvement by data augmentation (e.g., synthesis of selection patterns for each emotional state) and regularization (e.g., dropout). The output of the AI model is also linked to subsequent reservation processing and the feedback unit, and is utilized for personalization of the entire clinic selection experience. As a result, the selection unit achieves high-dimensional emotion feature extraction, nonlinear optimization, and real-time adaptive processing without relying on simple human template switching or heuristics, and provides optimized clinic selection criteria for each patient, resulting in improved selection accuracy, reduced selection burden, and maximized patient satisfaction. Application fields include medical institution recommendation, telemedicine platforms, personalized healthcare, and customer support reception. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of selection criteria adjustment, automatic optimization of recommendation quality, reduction of communication load, etc.).

The selection unit can improve the accuracy of selection by considering the interrelationship of the patient's symptoms when selecting a clinic. For example, when the patient has multiple symptoms, the selection unit prioritizes proposing clinics that can address each symptom. When the patient's symptoms are related, the selection unit can propose clinics that can address the related symptoms. When the patient's symptoms are independent, the selection unit can propose clinics for each symptom individually. Thus, the selection unit can improve the accuracy of clinic selection by considering the interrelationship of the patient's symptoms. Some or all of the above-described processing in the selection unit may be performed using AI or without using AI. For example, the selection unit can use the interrelationship of the patient's symptoms as input and select clinics using an AI model to improve selection accuracy. Specifically, the selection unit preprocesses multiple symptom data received from the reception unit (e.g., symptom labels, inter-symptom relevance scores, symptom onset time series, comorbidity risk, etc.) and clinic-specific symptom lists, specialty scores, treatment performance data obtained from the clinic database as multidimensional vectors or graph-structured data. The selection unit inputs these symptom relevance data and clinic information into a symptom compatibility analysis AI model (e.g., graph neural network, multitask learning model, Transformer-based large language model, etc.) to estimate clinic compatibility scores for each symptom and the priority of clinics capable of simultaneously addressing multiple symptoms. Examples of AI model inputs include (1) “Symptom A=low back pain, Symptom B=stiff shoulders, relevance=0.8, onset date A=2024-05-01, onset date B=2024-04-15, Clinic X: low back pain compatible=yes, stiff shoulders compatible=yes, specialty=high” and (2) “Symptom A=low back pain, Symptom B=sleep disorder, relevance=0.3, high independence, Clinic Y: low back pain compatible=yes, sleep disorder compatible=no”. The selection unit generates, as outputs of the AI model, candidate clinic lists (e.g., clinic ID, multi-symptom compatibility score, recommendation reason, specialty, distance, etc.) and individual recommendation lists for each symptom. For example, outputs such as “Clinic A: multi-symptom compatibility score 0.92, high specialty” or “Clinic B: Symptom A compatibility score 0.85, Symptom B compatibility score 0.80” are generated. The selection unit optimizes clinic selection accuracy within the AI model using loss functions (e.g., multi-label classification loss, relevance estimation loss, ranking loss) and updates weights by gradient descent. Furthermore, the patient's past selection history and feedback data are reflected by online learning or reinforcement learning to continuously improve recommendation accuracy. The output of the AI model is presented to the patient, who can select a clinic from the list and make reservations or inquiries. As a result, the selection unit achieves high-dimensional symptom relevance extraction, nonlinear optimization, and real-time adaptive processing without relying on simple human symptom listing or heuristics, and provides improved clinic selection accuracy considering multiple symptoms and symptom relevance, maximized patient satisfaction, and efficient use of medical resources. Application fields include medical institution recommendation, telemedicine platforms, multiple disease management support, and personalized healthcare in general. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of multi-symptom recommendation, automatic optimization of recommendation quality, reduction of communication load, etc.).

The selection unit can perform selection by considering the patient's attribute information when selecting a clinic. For example, the selection unit proposes appropriate clinics based on the patient's age or gender. The selection unit can also propose appropriate clinics based on the patient's lifestyle or occupation. The selection unit can also propose appropriate clinics based on the patient's past treatment history. Thus, the selection unit can select clinics by considering the patient's attribute information. Some or all of the above-described processing in the selection unit may be performed using AI or without using AI. For example, the selection unit can use the patient's attribute information as input and select clinics using an AI model for clinic selection. Specifically, the selection unit preprocesses the patient's attribute information received from the reception unit (e.g., age, gender, occupation, lifestyle, medical history, treatment history, etc.) and clinic-specific specialty, patient group support record, facility information obtained from the clinic database as multidimensional or categorical vectors. The selection unit inputs these attribute information and clinic information into an attribute compatibility analysis AI model (e.g., multilayer perceptron, content-based recommendation model, Transformer-based large language model, etc.) to calculate matching scores between patient attributes and clinic characteristics. Examples of AI model inputs include (1) “Age=65, gender=female, occupation=office worker, lifestyle=little exercise, treatment history=chiropractic, acupuncture, Clinic X: elderly support=yes, female specialty=yes, exercise therapy equipment=no” and (2) “Age=35, gender=male, occupation=sales, lifestyle=sports enthusiast, treatment history=exercise therapy, Clinic Y: sports injury support=yes, young support=yes”. The selection unit generates, as outputs of the AI model, candidate clinic lists (e.g., clinic ID, attribute compatibility score, recommendation reason, specialty, facilities, etc.). For example, outputs such as “Clinic A: attribute compatibility score 0.95, female specialty, well-equipped” or “Clinic B: attribute compatibility score 0.88, sports injury support” are generated. The selection unit optimizes clinic selection accuracy within the AI model using loss functions (e.g., attribute compatibility classification loss, ranking loss) and updates weights by gradient descent. Furthermore, the patient's past selection history and feedback data are reflected by online learning or reinforcement learning to continuously improve recommendation accuracy. The output of the AI model is presented to the patient, who can select a clinic from the list and make reservations or inquiries. As a result, the selection unit achieves high-dimensional attribute feature extraction, nonlinear optimization, and real-time adaptive processing without relying on simple human attribute filters or heuristics, and provides improved selection accuracy and maximized patient satisfaction through optimized clinic selection for each patient, as well as efficient use of medical resources. Application fields include medical institution recommendation, telemedicine platforms, and personalized healthcare in general. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of attribute compatibility recommendation, automatic optimization of recommendation quality, reduction of communication load, etc.).

The selection unit can estimate the patient's emotions and adjust the order in which clinic selection results are displayed based on the estimated emotions. For example, when the patient is relaxed, the selection unit displays clinics in an order that includes detailed information. When the patient is feeling stressed, the selection unit can display clinics in an order that includes simple information. When the patient is in a hurry, the selection unit can prioritize displaying the highest-rated clinics. Thus, the selection unit can adjust the display order of clinic selection results according to the patient's emotions. Emotion estimation is implemented, for example, by using an emotion estimation function with an emotion engine or generative AI. Generative AI may include text generation AI (e.g., LLM) or multimodal generative AI, but is not limited to these examples. Some or all of the above-described processing in the selection unit may be performed using AI or without using AI. For example, the selection unit can use the patient's emotion data as input and adjust the display order using an AI model for display order control. Specifically, the selection unit preprocesses the patient's emotion data (e.g., scores for stress level, relaxation level, urgency, emotion labels, emotion estimation time-series data, etc.) received from the reception unit or feedback unit, and candidate clinic lists (e.g., clinic ID, evaluation score, amount of detailed information, etc.) as multidimensional vectors. The selection unit integrates these emotion data with candidate clinic information and inputs them into a display order control AI model (e.g., ranking learning model, Transformer-based large language model, conditional recommendation network, etc.). Examples of AI model inputs include (1) “Emotion: stress level 0.85, relaxation level 0.10, urgency 0.05; number of candidate clinics=10; evaluation score distribution=0.9 to 0.7” and (2) “Emotion: relaxation level 0.90, stress level 0.05; number of candidate clinics=5; amount of detailed information=high”. The selection unit generates, as outputs of the AI model, display order parameters (e.g., evaluation order, detailed information priority order, simple display order, etc.) and actual candidate clinic lists (e.g., display only top 3, display all with details, etc.). For example, outputs such as “display order: evaluation order, top 3 only” or “display order: detailed information priority, display all” are generated. The selection unit optimizes in real time the display order and information amount of the candidate clinic list on the patient's device or web interface based on the AI model output. Furthermore, the patient's emotion estimation results and selection behavior history are used for online learning or reinforcement learning to update the AI model weights, continuously improving personalization accuracy. Internal processing of the AI model includes optimization of display order control accuracy using loss functions (e.g., ranking loss, cross-entropy loss), learning by gradient descent of weights, and implementation of generalization performance improvement by data augmentation (e.g., synthesis of display patterns for each emotional state) and regularization (e.g., dropout). The output of the AI model is also linked to subsequent reservation processing and the feedback unit, and is utilized for personalization of the entire clinic selection experience. As a result, the selection unit achieves high-dimensional emotion feature extraction, nonlinear optimization, and real-time adaptive processing without relying on simple human template switching or heuristics, and provides optimized display order of clinic selection results for each patient, resulting in improved selection accuracy, reduced selection burden, and maximized patient satisfaction. Application fields include medical institution recommendation, telemedicine platforms, personalized healthcare, and customer support reception. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of display order control, automatic optimization of recommendation quality, reduction of communication load, etc.).

The selection unit can perform selection by considering the patient's geographic distribution when selecting a clinic. For example, the selection unit prioritizes proposing nearby clinics based on the patient's residential area. The selection unit can also propose clinics at the travel destination when the patient is traveling. The selection unit can also propose clinics in the new residential area when the patient is planning to move. Thus, the selection unit can select clinics by considering the patient's geographic distribution. Some or all of the above-described processing in the selection unit may be performed using AI or without using AI. For example, the selection unit can use the patient's geographic distribution as input and select clinics using an AI model for clinic selection. Specifically, the selection unit preprocesses the patient's geographic location information received from the reception unit (e.g., GPS coordinates, address, region estimated from IP address, travel plans, planned relocation area, etc.) and clinic-specific location, access information, regional characteristic data obtained from the clinic database as multidimensional or categorical vectors. The selection unit inputs these geographic distribution data and clinic information into a geographic information-linked AI model (e.g., multilayer perceptron with geographic clustering, geographic recommendation model, multimodal neural network, etc.) to generate a list of optimal candidate clinics for the patient's current or planned location. Examples of AI model inputs include (1) “Current location=Chuo-ku, Tokyo; travel plan=Kita-ku, Osaka; Clinic X: location=Chuo-ku; Clinic Y: location=Kita-ku” and (2) “Planned relocation area=Nishi-ku, Yokohama; Clinic Z: location=Nishi-ku”. The selection unit generates, as outputs of the AI model, candidate clinic lists (e.g., clinic ID, geographic compatibility score, recommendation reason, distance, access information, etc.). For example, outputs such as “Clinic A: geographic compatibility score 0.98, distance 0.5 km” or “Clinic B: geographic compatibility score 0.90, travel destination support” are generated. The selection unit optimizes clinic selection accuracy within the AI model using loss functions (e.g., geographic compatibility classification loss, ranking loss) and updates weights by gradient descent. Furthermore, the patient's past selection history, movement history, and feedback data are reflected by online learning or reinforcement learning to continuously improve recommendation accuracy. The output of the AI model is presented to the patient, who can select a clinic from the list and make reservations or inquiries. As a result, the selection unit achieves high-dimensional geographic information extraction, nonlinear optimization, and real-time adaptive processing without relying on simple human geographic search or heuristics, and provides improved selection accuracy and maximized patient satisfaction through optimized clinic selection for each patient, as well as efficient use of medical resources. Application fields include medical institution recommendation, telemedicine platforms, traveler health management, and personalized healthcare in general. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of geographic recommendation, automatic optimization of recommendation quality, reduction of communication load, etc.).

The selection unit can improve the accuracy of selection by referring to related literature of the patient when selecting a clinic. For example, the selection unit refers to medical literature related to the patient's symptoms and proposes appropriate clinics. The selection unit can also refer to literature related to the patient's past treatment history and propose appropriate clinics. The selection unit can also refer to literature related to the patient's lifestyle and propose appropriate clinics. Thus, the selection unit can improve the accuracy of clinic selection by referring to related literature of the patient. Some or all of the above-described processing in the selection unit may be performed using AI or without using AI. For example, the selection unit can use the patient's related literature as input and select clinics using an AI model to improve selection accuracy. Specifically, the selection unit preprocesses the patient's symptom data (e.g., symptom labels, onset date, treatment history, lifestyle, etc.) and abstracts of papers, treatment recommendation guidelines, case reports, and clinical performance data of clinics obtained from related medical literature databases as text embedding vectors or multidimensional vectors. The selection unit inputs these patient data and literature information into a literature-referencing AI model (e.g., BERT-based text classification model, content-based recommendation model, multimodal neural network, etc.) to estimate the optimal treatment method or clinic type for the patient's symptoms, treatment history, and lifestyle, and generate a candidate clinic list. Examples of AI model inputs include (1) “Symptom=low back pain, treatment history=acupuncture, chiropractic, lifestyle=desk work, related literature: exercise therapy recommended for low back pain” and (2) “Symptom=herniated disc, treatment history=physical therapy, related literature: case report of surgical indication”. The selection unit generates, as outputs of the AI model, candidate clinic lists (e.g., clinic ID, literature compatibility score, recommendation reason, treatment method basis, etc.). For example, outputs such as “Clinic A: literature compatibility score 0.93, exercise therapy recommended” or “Clinic B: literature compatibility score 0.88, abundant surgical experience” are generated. The selection unit optimizes clinic selection accuracy within the AI model using loss functions (e.g., text classification loss, ranking loss) and updates weights by gradient descent. Furthermore, the patient's past selection history and feedback data are reflected by online learning or reinforcement learning to continuously improve recommendation accuracy. The output of the AI model is presented to the patient, who can select a clinic from the list and make reservations or inquiries. As a result, the selection unit achieves high-dimensional literature information extraction, nonlinear optimization, and real-time adaptive processing without relying on simple human literature referencing or heuristics, and provides improved selection accuracy and maximized patient satisfaction through optimized clinic selection for each patient, as well as efficient use of medical resources. Application fields include medical institution recommendation, telemedicine platforms, evidence-based medical support, and personalized healthcare in general. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of literature-referencing recommendation, automatic optimization of recommendation quality, reduction of communication load, etc.).

The feedback unit can estimate the patient's emotions and adjust the method of displaying feedback based on the estimated emotions. For example, when the patient is relaxed, the feedback unit provides detailed feedback. When the patient is feeling stressed, the feedback unit can provide simple and concise feedback. When the patient is in a hurry, the feedback unit can provide feedback that can be quickly understood. Thus, the feedback unit can adjust the method of displaying feedback according to the patient's emotions. Emotion estimation is implemented, for example, by using an emotion estimation function with an emotion engine or generative AI. Generative AI may include text generation AI (e.g., LLM) or multimodal generative AI, but is not limited to these examples. Some or all of the above-described processing in the feedback unit may be performed using AI or without using AI. For example, the feedback unit can use the patient's emotion data as input and adjust the method of displaying feedback using an AI model for feedback display control. Specifically, the feedback unit preprocesses the patient's emotion data (e.g., scores for stress level, relaxation level, urgency, emotion labels, emotion estimation time-series data, etc.) received from the reception unit or generation unit as multidimensional vectors. The feedback unit integrates these emotion data with treatment effect data, treatment plan history, and patient feedback history, and inputs them into a feedback display control AI model (e.g., Transformer-based large language model, conditional generation network, multimodal neural network, etc.). Examples of AI model inputs include (1) “Emotion: stress level 0.85, relaxation level 0.10, urgency 0.05; treatment effect: pain score 6→3, satisfaction 4, comment ‘Low back pain was alleviated after treatment’” and (2) “Emotion: relaxation level 0.90, stress level 0.05; treatment effect: pain score 3→2, satisfaction 5, comment ‘Felt lighter after treatment’”. The feedback unit generates, as outputs of the AI model, feedback display parameters (e.g., detail level: high/medium/low, summarization rate, number of display items, natural language generation template selection flag, etc.) and actual feedback text (e.g., “Pain was reduced by 3 points after this treatment. Continued stretching is recommended” or “Treatment effect: good. Next time, let's add exercise therapy”). The AI model internally uses loss functions (e.g., display detail classification loss, natural language generation loss) to optimize the generation accuracy of feedback display methods tailored to the patient's emotional state by optimizing weights via gradient descent. Furthermore, patient feedback (e.g., feedback comprehensibility evaluation, satisfaction with display amount, etc.) is reflected by online learning or reinforcement learning to continuously improve the model's personalization accuracy. The output of the AI model is displayed in real time on the patient's device or web interface, allowing the patient to receive optimal feedback explanations tailored to their emotional state. As a technical effect, the feedback unit achieves high-dimensional emotion feature extraction, nonlinear optimization, and real-time adaptive processing without relying on simple human template switching or heuristics, and provides optimized feedback display for each patient, resulting in improved understanding of treatment effects, reduced explanation burden, and maximized patient satisfaction. Application fields include medical feedback management, personalized healthcare, telemedicine platforms, and adaptive feedback generation in education. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of feedback display generation, automatic optimization of explanation quality, reduction of communication load, etc.).

The feedback unit can optimize current feedback by referring to past feedback data during feedback. For example, the feedback unit evaluates the progress of current treatment based on the patient's past feedback data. The feedback unit can also predict the effect of treatment from past feedback data and provide current feedback. The feedback unit can also analyze past feedback data and propose improvements to the current treatment plan. Thus, the feedback unit can optimize current feedback by referring to past feedback data. Some or all of the above-described processing in the feedback unit may be performed using AI or without using AI. For example, the feedback unit can use past feedback data as input and provide feedback using an AI model for feedback optimization. Specifically, the feedback unit preprocesses past feedback data accumulated for each patient (e.g., pain scores before and after treatment, satisfaction, free-text comments, treatment plan history, clinic history, time-series changes in treatment effect, etc.) as time-series vectors or text embedding vectors. The feedback unit inputs these data into a feedback optimization AI model (e.g., LSTM for time-series analysis, multilayer perceptron for history pattern classification, content-based recommendation model, Transformer-based large language model, etc.) to generate current treatment progress evaluation, treatment effect prediction, treatment plan improvement proposals, and patient feedback messages. Examples of AI model inputs include (1) “Pain scores for the last three sessions: 6→4→3, satisfaction: 3→4→5, treatment: stretching, massage” and (2) “Past comments: ‘Low back pain was alleviated after treatment’, ‘Exercise therapy was effective’, clinic history: chiropractic clinic→orthopedic clinic”. The feedback unit generates, as outputs of the AI model, current treatment progress evaluation (e.g., improvement trend, stagnation, deterioration), treatment effect prediction (e.g., predicted pain score after next treatment), treatment plan improvement proposals (e.g., increase stretching frequency, recommend changing clinic, etc.), and patient feedback messages (e.g., “Treatment effect is stable, so continuation of the current plan is recommended”) as structured data or natural language text. The AI model internally uses loss functions (e.g., time-series prediction loss, classification loss, natural language generation loss) to optimize the accuracy of feedback optimization based on past data by optimizing weights via gradient descent. Furthermore, new patient feedback and treatment effect data are reflected by online learning or reinforcement learning to continuously improve the model's personalization accuracy. The output of the AI model is displayed in real time on the patient's device or web interface, allowing the patient to receive optimal feedback based on their treatment history. As a technical effect, the feedback unit achieves high-dimensional history feature extraction, nonlinear optimization, and real-time adaptive processing without relying on simple human history referencing or heuristics, and provides optimized feedback for each patient, resulting in maximized treatment effect, accelerated treatment plan improvement, and improved patient satisfaction. Application fields include medical feedback management, personalized healthcare, telemedicine platforms, and chronic disease management. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of history-referenced feedback generation, automatic optimization of explanation quality, reduction of communication load, etc.).

The feedback unit can apply different feedback methods for each category of the patient's symptoms during feedback. For example, in the case of low back pain due to muscle tension, the feedback unit provides feedback on the effects of stretching and massage. In the case of low back pain due to herniated disc, the feedback unit can provide feedback on the effects of physical therapy or surgery. In the case of chronic low back pain, the feedback unit can provide feedback on the effects of long-term rehabilitation. Thus, the feedback unit can apply different feedback methods for each category of the patient's symptoms. Some or all of the above-described processing in the feedback unit may be performed using AI or without using AI. For example, the feedback unit can use the category of the patient's symptoms as input and provide feedback using an AI model that applies different feedback methods. Specifically, the feedback unit preprocesses the patient's symptom data (e.g., symptom category label: muscular, disc-related, chronic, etc.; symptom feature vector; image diagnosis result vector, etc.) received from the reception unit or generation unit as multidimensional vectors, and estimates the symptom category using a symptom category classification AI model (e.g., convolutional neural network for image classification, BERT-based text classification, multimodal fusion model, etc.). The feedback unit selects the optimal feedback generation AI model (e.g., model for muscular low back pain, model for disc-related low back pain, model for chronic low back pain, etc.) using a feedback method selection module (e.g., rule-based branching, conditional generation network, neural network for algorithm selection, etc.) according to the estimated symptom category, and generates feedback. Examples of AI model inputs include (1) “Symptom category: muscular, low back pain score=7, treatment: stretching, massage” and (2) “Symptom category: disc-related, image diagnosis: herniation observed, treatment: physical therapy, surgery”. The feedback unit generates, as outputs of the AI model, feedback content (e.g., evaluation of stretching effect, evaluation of physical therapy effect, evaluation of rehabilitation effect, etc.), recommended treatment continuation level, and patient feedback messages (e.g., “Continued stretching is relieving muscle tension”, “Physical therapy is showing effects”) as structured data or natural language text. The AI model internally uses loss functions (e.g., category classification loss, effect prediction loss, natural language generation loss) to optimize the accuracy of feedback generation for each symptom category by optimizing weights via gradient descent. Furthermore, patient feedback and treatment effect data are reflected by online learning or reinforcement learning to continuously improve the model's personalization accuracy. The output of the AI model is displayed in real time on the patient's device or web interface, allowing the patient to receive feedback optimized for their symptom category. As a technical effect, the feedback unit achieves high-dimensional symptom feature extraction, nonlinear optimization, and real-time adaptive processing without relying on simple human rule-based branching or heuristics, and provides optimized feedback for each symptom category, resulting in maximized treatment effect, reduced explanation burden, and improved patient satisfaction. Application fields include medical feedback management, personalized healthcare, telemedicine platforms, and disease-specific treatment support systems. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., automation of feedback method selection, computational efficiency, automatic optimization of explanation quality, etc.).

The feedback unit can estimate the patient's emotions and adjust the importance of feedback based on the estimated emotions. For example, when the patient is relaxed, the feedback unit provides detailed feedback. When the patient is feeling stressed, the feedback unit can provide feedback focused on important points. When the patient is in a hurry, the feedback unit can provide feedback that can be quickly understood. Thus, the feedback unit can adjust the importance of feedback according to the patient's emotions. Emotion estimation is implemented, for example, by using an emotion estimation function with an emotion engine or generative AI. Generative AI may include text generation AI (e.g., LLM) or multimodal generative AI, but is not limited to these examples. Some or all of the above-described processing in the feedback unit may be performed using AI or without using AI. For example, the feedback unit can use the patient's emotion data as input and adjust the importance of feedback using an AI model for feedback importance control. Specifically, the feedback unit preprocesses the patient's emotion data (e.g., scores for stress level, relaxation level, urgency, emotion labels, emotion estimation time-series data, etc.) received from the reception unit or generation unit as multidimensional vectors. The feedback unit integrates these emotion data with treatment effect data, treatment plan history, and patient feedback history, and inputs them into a feedback importance control AI model (e.g., Transformer-based large language model, conditional generation network, multimodal neural network, etc.). Examples of AI model inputs include (1) “Emotion: stress level 0.85, relaxation level 0.10, urgency 0.05; treatment effect: pain score 6→3, satisfaction 4, comment ‘Low back pain was alleviated after treatment’” and (2) “Emotion: relaxation level 0.90, stress level 0.05; treatment effect: pain score 3→2, satisfaction 5, comment ‘Felt lighter after treatment’”. The feedback unit generates, as outputs of the AI model, feedback importance parameters (e.g., importance: high/medium/low, summarization rate, number of display items, natural language generation template selection flag, etc.) and actual feedback text (e.g., “Pain was reduced by 3 points after this treatment. Continued stretching is recommended” or “Treatment effect: good. Next time, let's add exercise therapy”). The AI model internally uses loss functions (e.g., importance classification loss, natural language generation loss) to optimize the generation accuracy of feedback importance tailored to the patient's emotional state by optimizing weights via gradient descent. Furthermore, patient feedback (e.g., feedback comprehensibility evaluation, satisfaction with display amount, etc.) is reflected by online learning or reinforcement learning to continuously improve the model's personalization accuracy. The output of the AI model is displayed in real time on the patient's device or web interface, allowing the patient to receive optimal feedback explanations tailored to their emotional state. As a technical effect, the feedback unit achieves high-dimensional emotion feature extraction, nonlinear optimization, and real-time adaptive processing without relying on simple human template switching or heuristics, and provides optimized feedback importance for each patient, resulting in improved understanding of treatment effects, reduced explanation burden, and maximized patient satisfaction. Application fields include medical feedback management, personalized healthcare, telemedicine platforms, and adaptive feedback generation in education. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of feedback importance control, automatic optimization of explanation quality, reduction of communication load, etc.).

The feedback unit can analyze changes in feedback based on the onset timing of the patient's symptoms during feedback. For example, for recently developed symptoms, the feedback unit provides prompt feedback. For symptoms that have persisted for a long time, the feedback unit can provide long-term feedback. For recurrent symptoms, the feedback unit can provide feedback for recurrence prevention. Thus, the feedback unit can analyze changes in feedback based on the onset timing of the patient's symptoms. Some or all of the above-described processing in the feedback unit may be performed using AI or without using AI. For example, the feedback unit can use the onset timing of the patient's symptoms as input and provide feedback using an AI model for feedback change analysis. Specifically, the feedback unit preprocesses the patient's symptom data (e.g., onset timing, symptom duration, number of recurrences, past treatment history, etc.) received from the reception unit or generation unit, and past feedback history (e.g., pain scores before and after treatment, satisfaction, treatment effect trends, etc.) as multidimensional vectors or time-series tensors. The feedback unit inputs these data into an onset timing-linked feedback analysis AI model (e.g., LSTM for time-series analysis, multilayer perceptron for onset timing feature extraction, content-based recommendation model, Transformer-based large language model, etc.) to analyze changes in feedback content and treatment effect trends, and generate optimal feedback. Examples of AI model inputs include (1) “Onset timing=2024-06-01, duration=3 days, novelty=high, treatment history=none, past feedback: none” and (2) “Onset timing=2023-01-01, duration=1 year, chronicity=high, treatment history=multiple times, past feedback: pain score 7→5→4”. The feedback unit generates, as outputs of the AI model, feedback content (e.g., prompt response message for acute symptoms, long-term advice for chronic symptoms, recurrence prevention recommendations, etc.), treatment effect trend graphs, and patient feedback messages (e.g., “Since this is an acute symptom, continued early treatment is recommended”, “Since this is a chronic symptom, long-term rehabilitation is recommended”, “For recurrence prevention, focus on lifestyle improvement”) as structured data or natural language text. The AI model internally uses loss functions (e.g., time-series prediction loss, classification loss, natural language generation loss) to optimize the generation accuracy of feedback tailored to onset timing by optimizing weights via gradient descent. Furthermore, new patient feedback and treatment effect data are reflected by online learning or reinforcement learning to continuously improve the model's personalization accuracy. The output of the AI model is displayed in real time on the patient's device or web interface, allowing the patient to receive optimal feedback according to the onset timing of their symptoms. As a technical effect, the feedback unit achieves high-dimensional time-series feature extraction, nonlinear optimization, and real-time adaptive processing without relying on simple human heuristics or rule-based judgment, and provides optimized feedback for each patient, resulting in improved timeliness of treatment initiation, maximized treatment effect, and improved patient satisfaction. Application fields include medical feedback management, personalized healthcare, telemedicine platforms, and chronic disease management. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of onset timing-linked feedback generation, automatic optimization of explanation quality, reduction of communication load, etc.).

The feedback unit can analyze feedback by referring to related market data of the patient during feedback. For example, the feedback unit provides feedback on the effect of treatment based on market data related to the patient's symptoms. The feedback unit can also refer to market data related to the patient's past treatment history and provide feedback. The feedback unit can also refer to market data related to the patient's lifestyle and provide feedback. Thus, the feedback unit can analyze feedback by referring to related market data of the patient. Some or all of the above-described processing in the feedback unit may be performed using AI or without using AI. For example, the feedback unit can use related market data of the patient as input and provide feedback using an AI model for market-referenced feedback analysis. Specifically, the feedback unit preprocesses the patient's symptom data (e.g., symptom labels, onset timing, treatment history, lifestyle, etc.) and market data obtained from related market databases (e.g., market trends of treatment methods, clinic popularity rankings, statistical data on treatment effects, usage trends of lifestyle improvement services, etc.) as multidimensional vectors, categorical vectors, or text embedding vectors. The feedback unit inputs these patient data and market data into a market-referenced feedback analysis AI model (e.g., content-based recommendation model, BERT-based text classification model, multimodal neural network, etc.) to estimate the optimal treatment method, clinic type, and lifestyle improvement proposals for the patient's symptoms, treatment history, and lifestyle, and generate feedback. Examples of AI model inputs include (1) “Symptom=low back pain, treatment history=acupuncture, chiropractic, lifestyle=desk work, market data: low back pain treatment method ranking, chiropractic clinic popularity” and (2) “Symptom=stiff shoulders, treatment history=exercise therapy, market data: exercise therapy effect statistics, usage rate of lifestyle improvement services”. The feedback unit generates, as outputs of the AI model, feedback content (e.g., recommendation of popular treatment methods in the market, recommendation of clinic type, lifestyle improvement proposals, etc.), treatment effect evaluation based on market data, and patient feedback messages (e.g., “The current treatment method is reported to be highly effective in the market”, “Use of lifestyle improvement services is recommended”) as structured data or natural language text. The AI model internally uses loss functions (e.g., text classification loss, ranking loss, natural language generation loss) to optimize the generation accuracy of feedback based on market data by optimizing weights via gradient descent. Furthermore, new patient feedback, treatment effect data, and market data updates are reflected by online learning or reinforcement learning to continuously improve the model's personalization accuracy. The output of the AI model is displayed in real time on the patient's device or web interface, allowing the patient to receive market data-linked feedback according to their symptoms, treatment history, and lifestyle. As a technical effect, the feedback unit achieves high-dimensional market information extraction, nonlinear optimization, and real-time adaptive processing without relying on simple human market referencing or heuristics, and provides optimized market-linked feedback for each patient, resulting in maximized treatment effect, rationalization of treatment method selection, and improved patient satisfaction. Application fields include medical feedback management, personalized healthcare, telemedicine platforms, evidence-based medical support, and health management applications. Furthermore, the internal processing of the AI model is based on specific algorithm design, eliminating black-box descriptions, and includes error minimization by loss functions, optimization by gradient descent of weights, and generalization performance improvement by data augmentation and regularization, thereby achieving improvements in computer technology itself (e.g., computational efficiency of market-referenced feedback generation, automatic optimization of explanation quality, reduction of communication load, etc.).

The system according to the embodiment is not limited to the above-described examples, and various modifications are possible, for example, as follows.

The reception unit can automatically acquire the patient's past treatment history when inputting symptoms or health information and supplement the input content. For example, the reception unit can automatically display the treatment details and effects previously received by the patient and provide more accurate information by comparing with the current symptoms. In addition, the reception unit can automatically generate questions related to the current symptoms based on the patient's past treatment history, thereby improving the accuracy of the information input by the patient. Furthermore, the reception unit can propose treatment options for the current symptoms based on the patient's past treatment history.

The generation unit can predict the effect of the treatment plan based on the patient's symptoms and health information. For example, the generation unit can analyze past treatment data and predict the effect of treatment for patients with similar symptoms. In addition, the generation unit can individually predict the effect of the treatment plan by considering the patient's lifestyle and health information. Furthermore, the generation unit can monitor the effect of the treatment plan in real time and modify the treatment plan as necessary.

The selection unit can narrow down clinic options based on the patient's symptoms and health information. For example, the selection unit prioritizes displaying clinics that can address the patient's symptoms, enabling the patient to select the optimal clinic. In addition, the selection unit can propose the optimal clinic to the patient based on clinic evaluations and reviews. Furthermore, the selection unit can customize clinic options by considering the patient's lifestyle and health information.

The feedback unit can monitor the progress of the patient's treatment in real time and evaluate the effect of the treatment. For example, the feedback unit can provide real-time feedback on the effect after the patient receives treatment and propose modifications to the treatment plan. In addition, the feedback unit can display the progress of the patient's treatment in graphs or charts, allowing the patient to visually confirm the effect of the treatment. Furthermore, the feedback unit can compare the progress of the patient's treatment with that of other patients and evaluate the effect of the treatment.

The generation unit can provide advice to maximize the effect of the treatment plan based on the patient's lifestyle and health information. For example, the generation unit provides advice on exercise and diet tailored to the patient's lifestyle to maximize the effect of the treatment plan. In addition, the generation unit can propose lifestyle improvements to maximize the effect of the treatment plan based on the patient's health information. Furthermore, the generation unit can provide customized advice to maximize the effect of the treatment plan by considering the patient's lifestyle and health information.

The reception unit can estimate the patient's emotions and adjust the method of inputting symptoms or health information based on the estimated emotions. For example, when the patient is feeling stressed, a simple interface is provided and the input procedure is minimized. When the patient is relaxed, detailed input options are provided and customizable input methods can be proposed. When the patient is in a hurry, voice input is prioritized to enable rapid input of symptoms or health information. Thus, the reception unit can adjust the method of inputting symptoms or health information according to the patient's emotions.

The generation unit can estimate the patient's emotions and adjust the method of expressing the treatment plan based on the estimated emotions. For example, when the patient is relaxed, the generation unit provides a treatment plan that includes detailed explanations. When the patient is feeling stressed, the generation unit can provide a treatment plan that is simple and focused on key points. When the patient is in a hurry, the generation unit can provide a treatment plan that can be quickly understood. Thus, the generation unit can adjust the method of expressing the treatment plan according to the patient's emotions.

The selection unit can estimate the patient's emotions and adjust the selection criteria for clinics based on the estimated emotions. For example, if the patient is relaxed, detailed information is provided to support the selection of a clinic. If the patient is feeling stressed, simple information is provided to enable quick selection of a clinic. If the patient is in a hurry, the clinic with the highest rating can be preferentially proposed. Thus, the selection unit can adjust the selection criteria for clinics according to the patient's emotions.

The feedback unit can estimate the patient's emotions and adjust the method of displaying feedback based on the estimated emotions. For example, if the patient is relaxed, detailed feedback is provided. If the patient is feeling stressed, simple and concise feedback can be provided. If the patient is in a hurry, feedback that can be quickly understood can be provided. Thus, the feedback unit can adjust the method of displaying feedback according to the patient's emotions.

The generation unit can estimate the patient's emotions and adjust the length of the treatment plan based on the estimated emotions. For example, if the patient is relaxed, a longer treatment plan including detailed explanations is provided. If the patient is feeling stressed, a short and concise treatment plan can be provided. If the patient is in a hurry, a short treatment plan that can be quickly understood can be provided. Thus, the generation unit can adjust the length of the treatment plan according to the patient's emotions.

The following is a brief description of the processing flow of Example of the Embodiment.
Step 1: The reception unit inputs the patient's symptoms and health information. The patient's symptoms and health information include medical history, allergy information, current symptoms, and the like. For example, the patient can input detailed information such as the degree of lower back pain, onset timing, past treatment history, and lifestyle.
Step 2: The generation unit uses AI to analyze the information input by the reception unit and generate an optimal treatment plan. The generation unit proposes a treatment plan centered on stretching and massage based on the patient's symptoms and health information. In addition, the generation unit can generate a treatment plan individually customized by considering the patient's lifestyle and past treatment history.
Step 3: The selection unit selects a clinic based on the treatment plan generated by the generation unit. The selection unit can select the optimal clinic from nearby clinics based on the treatment plan proposed by AI. Furthermore, by referring to clinic ratings and reviews, a more appropriate selection can be made.
Step 4: The feedback unit provides feedback on the progress of treatment at the clinic selected by the selection unit. The feedback unit enables the patient to provide feedback online regarding the effects and impressions after receiving treatment. This allows the effect of the treatment to be confirmed in real time and the treatment plan to be modified as necessary.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, generation unit, selection unit, and feedback unit is implemented by at least one of, for example, a smart device 14 and a data processing apparatus 12. For example, the reception unit is implemented by a reception device 38 of the smart device 14 and provides an interface for inputting a patient's symptoms or health information. The generation unit is implemented, for example, by a specific processing unit 290 of the data processing apparatus 12 and analyzes the patient's symptoms or health information using AI to generate an optimal treatment plan. The selection unit is implemented, for example, by a control unit 46A of the smart device 14 and selects an optimal clinic based on the generated treatment plan. The feedback unit is implemented, for example, by an output device 40 of the smart device 14 and enables feedback on the progress of treatment online. The correspondence between each unit and the devices or control units is not limited to the examples described above and various modifications are possible.

### Second Embodiment

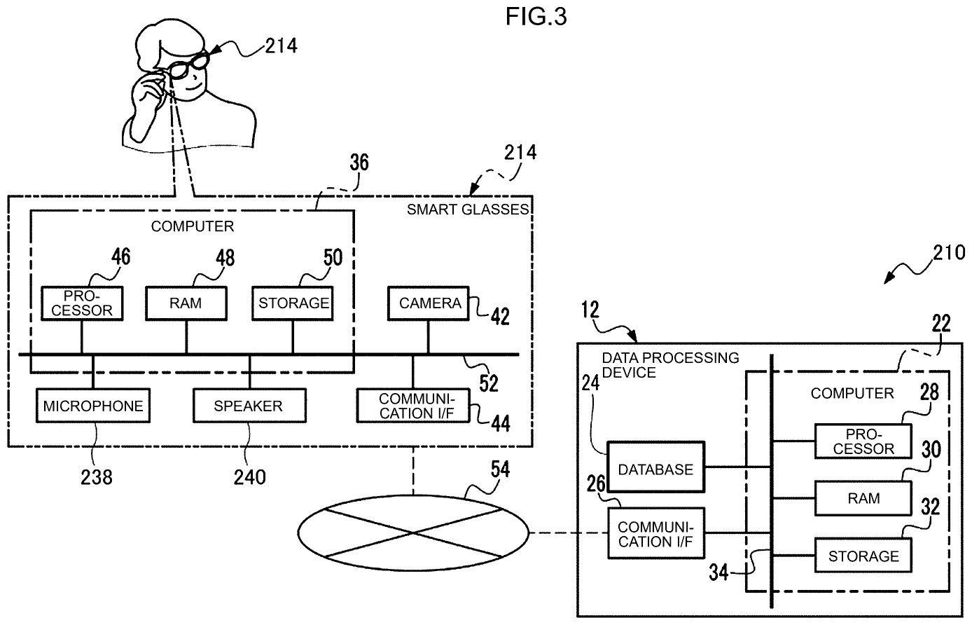

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

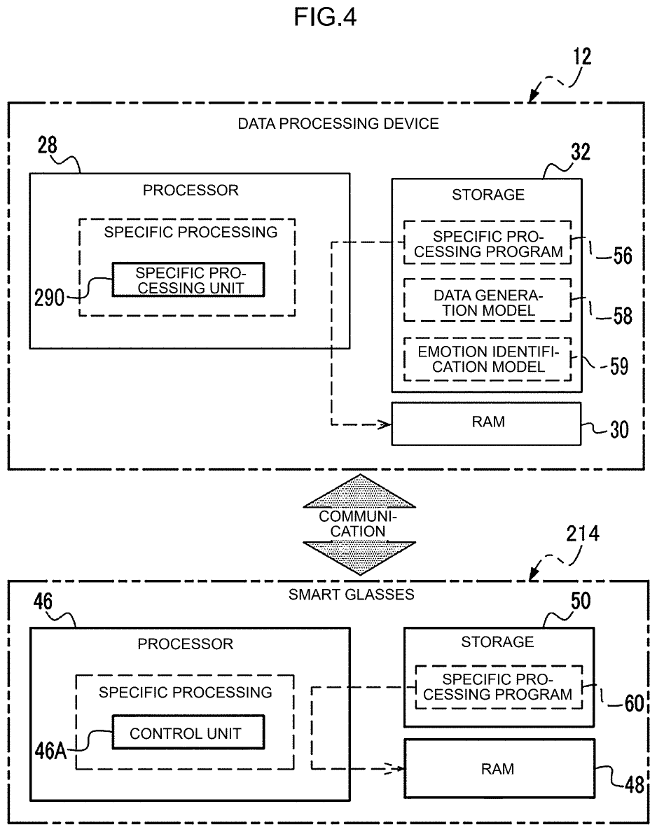

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, generation unit, selection unit, and feedback unit is implemented by at least one of, for example, smart glasses 214 and a data processing apparatus 12. For example, the reception unit is implemented by a microphone 238 of the smart glasses 214 and provides an interface for inputting a patient's symptoms or health information. The generation unit is implemented, for example, by a specific processing unit 290 of the data processing apparatus 12 and analyzes the patient's symptoms or health information using AI to generate an optimal treatment plan. The selection unit is implemented, for example, by a control unit 46A of the smart glasses 214 and selects an optimal clinic based on the generated treatment plan. The feedback unit is implemented, for example, by a speaker 240 of the smart glasses 214 and enables feedback on the progress of treatment online. The correspondence between each unit and the devices or control units is not limited to the examples described above and various modifications are possible.

### Third Embodiment

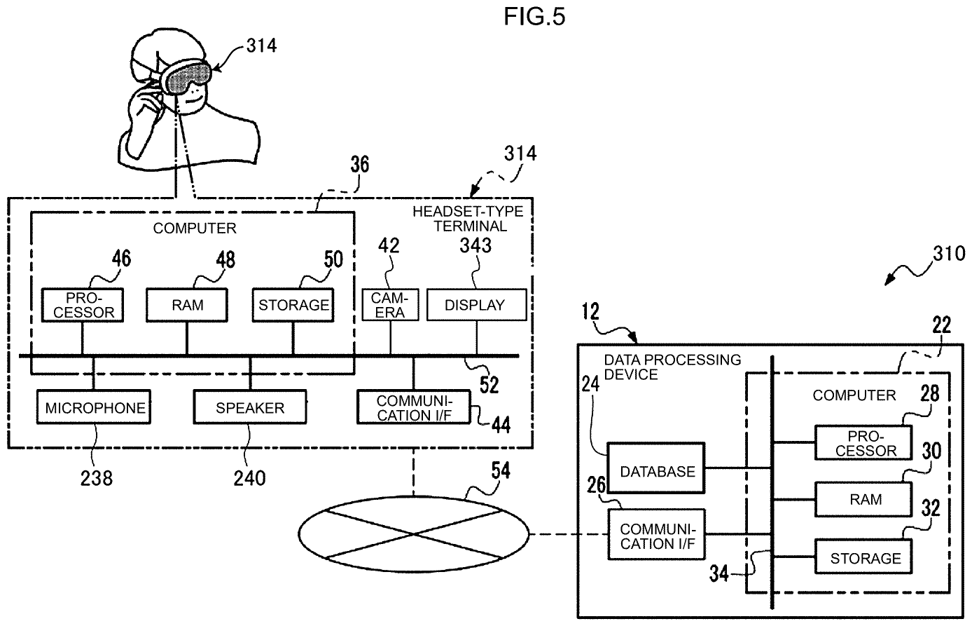

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

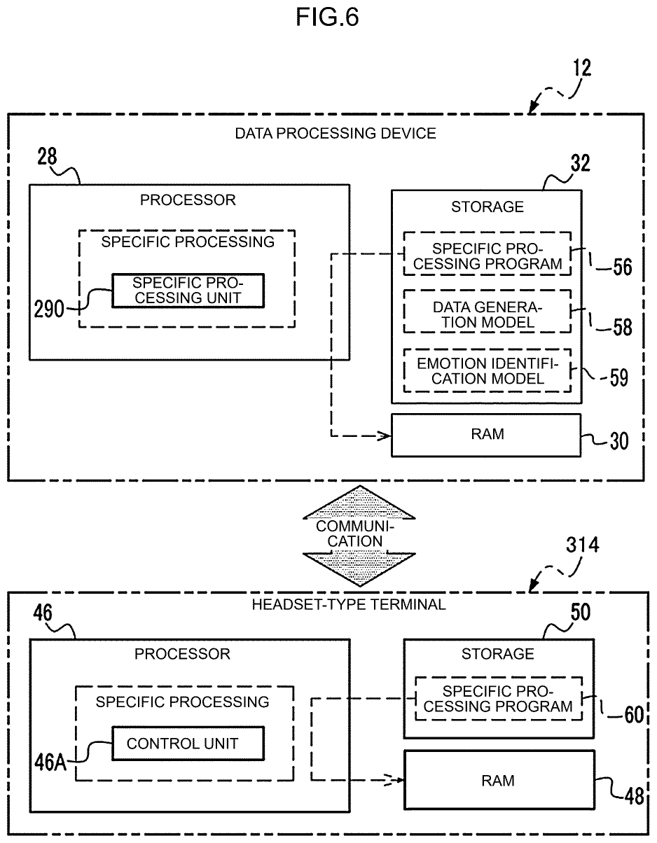

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, generation unit, selection unit, and feedback unit is implemented by at least one of, for example, a headset-type terminal 314 and a data processing apparatus 12. For example, the reception unit is implemented by a microphone 238 of the headset-type terminal 314 and provides an interface for inputting a patient's symptoms or health information. The generation unit is implemented, for example, by a specific processing unit 290 of the data processing apparatus 12 and analyzes the patient's symptoms or health information using AI to generate an optimal treatment plan. The selection unit is implemented, for example, by a control unit 46A of the headset-type terminal 314 and selects an optimal clinic based on the generated treatment plan. The feedback unit is implemented, for example, by a display 343 of the headset-type terminal 314 and enables feedback on the progress of treatment online. The correspondence between each unit and the devices or control units is not limited to the examples described above and various modifications are possible.

### Fourth Embodiment

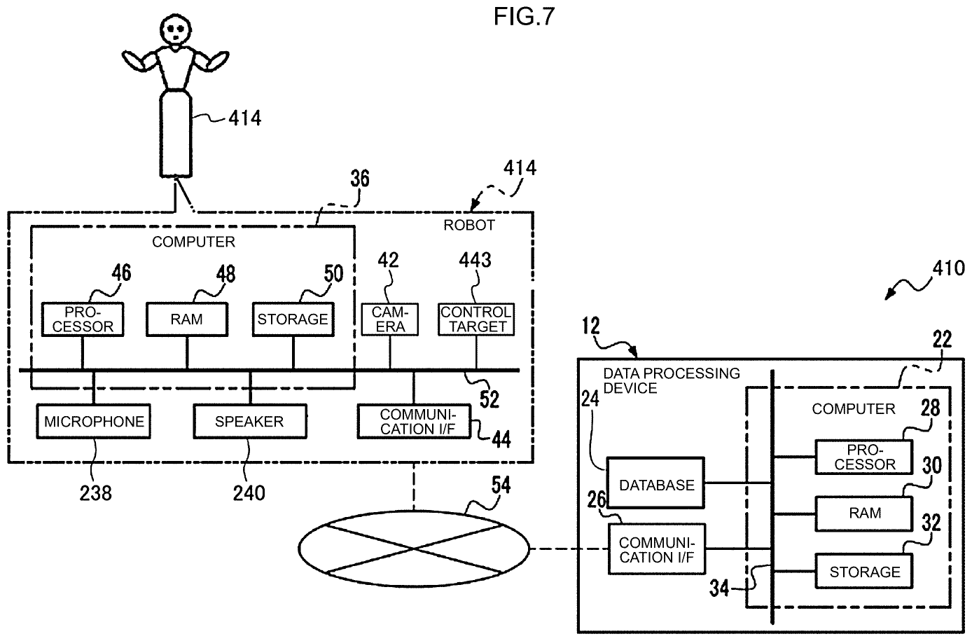

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

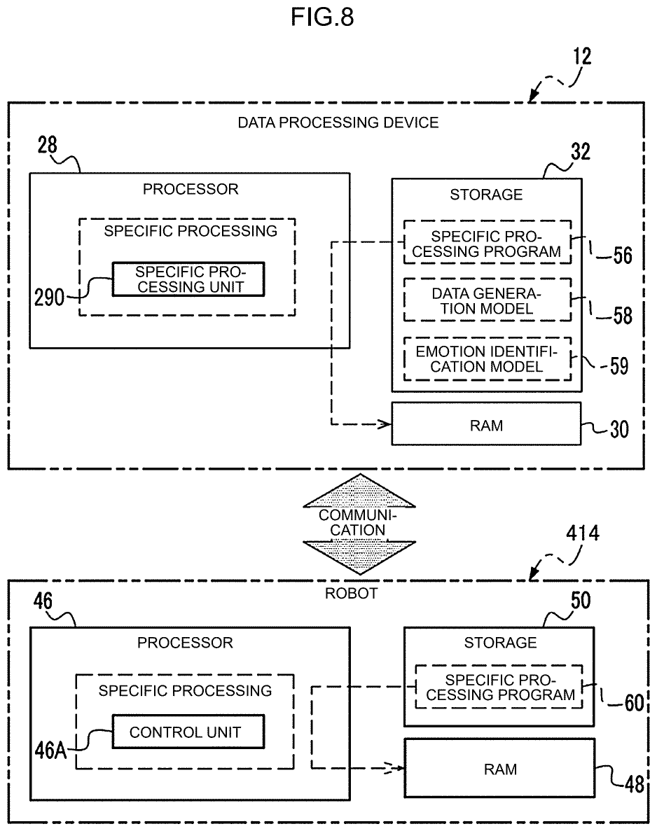

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, generation unit, selection unit, and feedback unit is implemented by at least one of, for example, a robot 414 and a data processing apparatus 12. For example, the reception unit is implemented by a microphone 238 of the robot 414 and provides an interface for inputting a patient's symptoms or health information. The generation unit is implemented, for example, by a specific processing unit 290 of the data processing apparatus 12 and analyzes the patient's symptoms or health information using AI to generate an optimal treatment plan. The selection unit is implemented, for example, by a control unit 46A of the robot 414 and selects an optimal clinic based on the generated treatment plan. The feedback unit is implemented, for example, by a speaker 240 of the robot 414 and enables feedback on the progress of treatment online. The correspondence between each unit and the devices or control units is not limited to the examples described above and various modifications are possible.

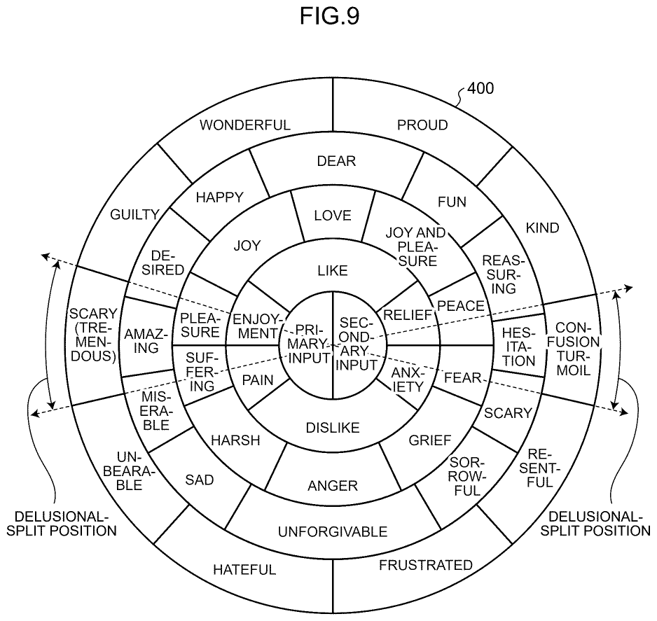

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

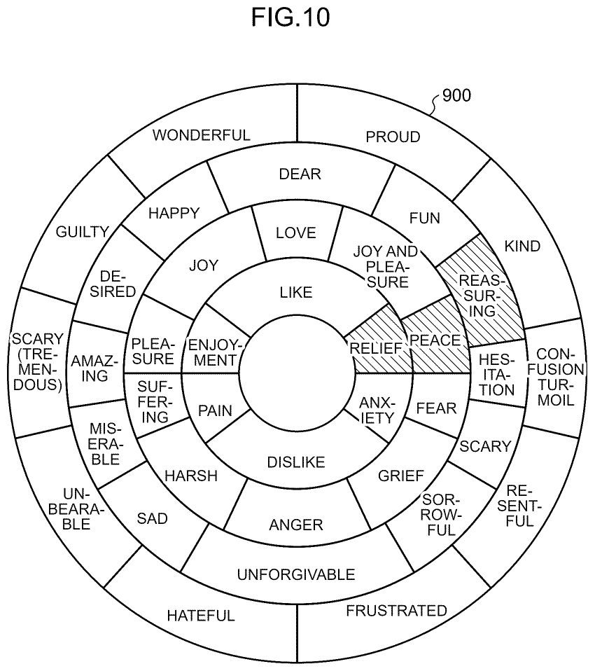

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Supplementary Note 1

A system comprising: a reception unit configured to input a patient's symptoms or health information; a generation unit configured to analyze the information input by the reception unit and generate a treatment plan; a selection unit configured to select a clinic based on the treatment plan generated by the generation unit; and a feedback unit configured to provide feedback on the progress of treatment at the clinic selected by the selection unit.

### Supplementary Note 2

The system according to Supplementary Note 1, wherein the generation unit is configured to generate an optimal treatment plan based on the patient's symptoms and health information.

### Supplementary Note 3

The system according to Supplementary Note 1, wherein the selection unit is configured to select a clinic based on the generated treatment plan.

### Supplementary Note 4

The system according to Supplementary Note 1, wherein the feedback unit is configured to share the progress of treatment in real time.

### Supplementary Note 5

The system according to Supplementary Note 1, wherein the generation unit is configured to customize the treatment plan based on the patient's lifestyle and past treatment history.

### Supplementary Note 6

The system according to Supplementary Note 1, wherein the feedback unit is configured to modify the treatment plan based on patient feedback.

### Supplementary Note 7

The system according to Supplementary Note 1, wherein the reception unit is configured to estimate the patient's emotions and adjust the method of inputting symptoms or health information based on the estimated emotions.

### Supplementary Note 8

The system according to Supplementary Note 1, wherein the reception unit is configured to analyze the patient's past symptom input history and propose an optimal input method.

### Supplementary Note 9

The system according to Supplementary Note 1, wherein the reception unit is configured to customize input items based on the patient's current living conditions and areas of interest when inputting symptoms or health information.

### Supplementary Note 10

The system according to Supplementary Note 1, wherein the reception unit is configured to estimate the patient's emotions and determine the priority of information to be input based on the estimated emotions.

### Supplementary Note 11

The system according to Supplementary Note 1, wherein the reception unit is configured to prioritize the input of highly relevant information by considering the patient's geographic location when inputting symptoms or health information.

### Supplementary Note 12

The system according to Supplementary Note 1, wherein the reception unit is configured to analyze the patient's social media activity and input relevant information when inputting symptoms or health information.

### Supplementary Note 13

The system according to Supplementary Note 1, wherein the generation unit is configured to estimate the patient's emotions and adjust the method of expressing the treatment plan based on the estimated emotions.

### Supplementary Note 14

The system according to Supplementary Note 1, wherein the generation unit is configured to adjust the level of detail of the treatment plan based on the importance of the patient's symptoms when generating the treatment plan.

### Supplementary Note 15

The system according to Supplementary Note 1, wherein the generation unit is configured to apply different treatment algorithms according to the category of the patient's symptoms when generating the treatment plan.

### Supplementary Note 16

The system according to Supplementary Note 1, wherein the generation unit is configured to estimate the patient's emotions and adjust the length of the treatment plan based on the estimated emotions.

### Supplementary Note 17

The system according to Supplementary Note 1, wherein the generation unit is configured to determine the priority of the treatment plan based on the onset timing of the patient's symptoms when generating the treatment plan.

### Supplementary Note 18

The system according to Supplementary Note 1, wherein the generation unit is configured to adjust the order of the treatment plan based on the relevance of the patient's symptoms when generating the treatment plan.

### Supplementary Note 19

The system according to Supplementary Note 1, wherein the selection unit is configured to estimate the patient's emotions and adjust the selection criteria for clinics based on the estimated emotions.

### Supplementary Note 20

The system according to Supplementary Note 1, wherein the selection unit is configured to improve the accuracy of selection by considering the interrelationship of the patient's symptoms when selecting a clinic.

### Supplementary Note 21

The system according to Supplementary Note 1, wherein the selection unit is configured to perform selection by considering the patient's attribute information when selecting a clinic.

### Supplementary Note 22

The system according to Supplementary Note 1, wherein the selection unit is configured to estimate the patient's emotions and adjust the order in which the selection results of clinics are displayed based on the estimated emotions.

### Supplementary Note 23

The system according to Supplementary Note 1, wherein the selection unit is configured to perform selection by considering the geographic distribution of the patient when selecting a clinic.

### Supplementary Note 24

The system according to Supplementary Note 1, wherein the selection unit is configured to improve the accuracy of selection by referring to related literature of the patient when selecting a clinic.

### Supplementary Note 25

The system according to Supplementary Note 1, wherein the feedback unit is configured to estimate the patient's emotions and adjust the method of displaying feedback based on the estimated emotions.

### Supplementary Note 26

The system according to Supplementary Note 1, wherein the feedback unit is configured to optimize current feedback by referring to past feedback data when providing feedback.

### Supplementary Note 27

The system according to Supplementary Note 1, wherein the feedback unit is configured to apply different feedback methods for each category of the patient's symptoms when providing feedback.

### Supplementary Note 28

The system according to Supplementary Note 1, wherein the feedback unit is configured to estimate the patient's emotions and adjust the importance of feedback based on the estimated emotions.

### Supplementary Note 29

The system according to Supplementary Note 1, wherein the feedback unit is configured to analyze changes in feedback based on the onset timing of the patient's symptoms when providing feedback.

### Supplementary Note 30

The system according to Supplementary Note 1, wherein the feedback unit is configured to analyze feedback by referring to related market data of the patient when providing feedback.

What is claimed is:

1. A system comprising:
a communication interface configured to communicate with a client terminal via a packet-switched network;
a memory storing a data generation model obtained by deep learning on a neural network, and an emotion identification model;
a database; and
circuitry configured to:
receive, from the client terminal via the communication interface, first structured data comprising at least one of text data, voice data, or image data;
estimate an emotion of a user by applying the emotion identification model to sensor data received from the client terminal;
analyze the first structured data using the data generation model to generate second structured data comprising at least one of a classification label, a recommendation text, or a priority score;
store the second structured data in the database;
receive, from the client terminal via the communication interface, query data;
retrieve, from the database, third structured data based on the query data and the estimated emotion;
generate, using the data generation model, inference data based on the retrieved third structured data; and
transmit the inference data to the client terminal via the communication interface and the packet-switched network, the inference data causing the client terminal to present the inference data to the user.

2. The system according to claim 1, wherein the first structured data comprises health information of the user, the health information comprising at least one of a symptom description, a pain score, an onset timing, a medical history, or an allergy information.

3. The system according to claim 1, wherein the second structured data comprises a treatment plan comprising at least one of a treatment content label, a recommended frequency, a recommended duration, or an estimated effect score.

4. The system according to claim 1, wherein the circuitry is further configured to generate a list of candidate entities based on the second structured data by calculating a matching score for each candidate entity using at least one of a ranking learning algorithm or a recommendation system comprising collaborative filtering.

5. The system according to claim 4, wherein the matching score is calculated based on at least one of a geographic distance, a specialty score, a rating score, or a congestion status associated with each candidate entity.

6. The system according to claim 1, wherein the circuitry is further configured to receive feedback data from the client terminal via the communication interface, the feedback data comprising at least one of an effect score, a satisfaction score, or a free-text comment, and to update the data generation model based on the feedback data using at least one of online learning or reinforcement learning.

7. The system according to claim 1, wherein the circuitry is further configured to adjust a method of receiving the first structured data based on the estimated emotion, such that when the estimated emotion indicates stress, the circuitry provides a simplified input interface, and when the estimated emotion indicates relaxation, the circuitry provides a detailed input interface.

8. The system according to claim 1, wherein the circuitry is further configured to analyze a past input history of the user stored in the database and propose an optimal input method based on the past input history using at least one of a time-series analysis model or a recommendation model.

9. The system according to claim 1, wherein the circuitry is further configured to customize input items displayed to the user based on at least one of a current interest or a current living condition of the user received from the client terminal.

10. The system according to claim 1, wherein the circuitry is further configured to determine a priority of information to be received based on the estimated emotion, such that when the estimated emotion indicates stress, the circuitry prioritizes receiving high-importance information.

11. The system according to claim 1, wherein the circuitry is further configured to receive geographic location information of the user from the client terminal and to prioritize receiving structured data associated with a geographic region corresponding to the geographic location information.

12. The system according to claim 1, wherein the circuitry is further configured to adjust an expression style of the second structured data based on the estimated emotion, such that when the estimated emotion indicates relaxation, the second structured data is generated in a detailed expression style, and when the estimated emotion indicates stress, the second structured data is generated in a concise expression style.

13. The system according to claim 1, wherein the circuitry is further configured to adjust a level of detail of the second structured data based on an importance score associated with the first structured data, such that for first structured data having a high importance score, detailed second structured data is generated.

14. The system according to claim 1, wherein the circuitry is further configured to apply different analysis algorithms according to a category of the first structured data.

15. The system according to claim 1, wherein the circuitry is further configured to adjust a length of the inference data based on the estimated emotion, such that when the estimated emotion indicates stress, the circuitry generates concise inference data, and when the estimated emotion indicates relaxation, the circuitry generates detailed inference data.

16. The system according to claim 1, wherein the circuitry is further configured to determine a priority of generating the second structured data based on a submission timing associated with the first structured data, such that first structured data having a more recent submission timing is processed with a higher priority.

17. The system according to claim 1, wherein the circuitry is further configured to analyze interrelationships among multiple items of the first structured data using at least one of a graph neural network or a multitask learning model, and to adjust an order of processing based on the analyzed interrelationships.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface, first structured data comprising at least one of text data input via the touch panel, voice data captured by the microphone, or image data captured by the camera;
estimate an emotion of a user by applying the emotion identification model to at least one of the voice data or the image data;
analyze the first structured data using the data generation model to generate second structured data comprising at least one of a classification label, a recommendation text, or a priority score;
store the second structured data in the database;
generate a list of candidate entities based on the second structured data by calculating a matching score for each candidate entity;
receive, from the client terminal via the communication interface, feedback data comprising at least one of an effect score or a satisfaction score;
adjust at least one of an expression style, a level of detail, or a length of inference data based on the estimated emotion;
generate, using the data generation model, the inference data based on the second structured data and the feedback data; and
transmit the inference data to the client terminal via the communication interface, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a data generation model obtained by deep learning on a neural network and an emotion identification model, and a database, the method comprising:
receiving, from a client terminal via the communication interface and a packet-switched network, first structured data comprising at least one of text data, voice data, or image data;
estimating an emotion of a user by applying the emotion identification model to sensor data received from the client terminal;
analyzing the first structured data using the data generation model to generate second structured data comprising at least one of a classification label, a recommendation text, or a priority score;
storing the second structured data in the database;
receiving, from the client terminal via the communication interface, query data;
retrieving, from the database, third structured data based on the query data and the estimated emotion;
generating, using the data generation model, inference data based on the retrieved third structured data; and
transmitting the inference data to the client terminal via the communication interface and the packet-switched network, the inference data causing the client terminal to present the inference data to the user.
